(19) United States
(12) Patent Application Publication
(10) Pub. No.: US 2015/0150910 A1
(43) Pub. Date: Jun. 4, 2015

(54) SUBSTITUTE THERAPY FOR GLUCOCORTICOIDS

(71) Applicant: WESTFAELISCHE WILHELMS-UNIVERSITAET MUENSTER, Munster (DE)

(72) Inventors: Jan EHRCHEN, Muenster (DE); Georg VARGA, Nordwalde (DE); Johannes ROTH, Muenster (DE); Cord SUNDERKOETTER, Muenster (DE)

(21) Appl. No.: 14/623,406

(22) Filed: Feb. 16, 2015

Related U.S. Application Data
(62) Division of application No. 13/637,195, filed on Sep. 25, 2012.

(30) Foreign Application Priority Data
Mar. 26, 2010 (EP) 10157919.1

(51) Int. Cl. A61K 35/15; A61K 45/06

(52) U.S. Cl. CPC A61K 35/15; A61K 45/06; A61K 2035/124

(57) ABSTRACT

The present invention relates to a pharmaceutical composition comprising glucocorticoid (GC)-induced human monocytes, and optionally a pharmaceutically acceptable carrier.

This application is a divisional of application Ser. No. 13/637,195, which was filed under U.S.C. §371 as the U.S. national phase of International Application No. PCT/EP2011/054630, filed Mar. 25, 2011, which designated the U.S. and claims the benefit of priority to European Patent Application No. 10157919.1 filed Mar. 26, 2010, which is hereby incorporated in its entirety including all tables, figures, and claims.

The instant application contains a Sequence Listing which has been submitted in ASCII format via EFS-Web and is hereby incorporated by reference in its entirety. Said ASCII copy, created on Feb. 16, 2015, is named SCH2100DV_Sequencelisting.txt and is 2 kilobytes in size.

The present invention relates to a pharmaceutical composition comprising glucocorticoid (GC)-induced human monocytes, and optionally a pharmaceutically acceptable carrier. The present invention further relates to a pharmaceutical composition comprising GC and optionally a pharmaceutically acceptable carrier, for use in the treatment of a disease which is GC-responsive, wherein said pharmaceutical composition is to be administered to human monocytes ex vivo. The pharmaceutical composition may be used for treatment of a disease which is GC-responsive. In another aspect, the present invention relates to the use of GC for the preparation of a pharmaceutical composition for the treatment of a patient in need of a GC-therapy, wherein said GC is to be administered to monocytes of said patient ex vivo. In a further embodiment, the present invention relates to a method for the preparation of a pharmaceutical composition comprising the step of contacting human monocytes ex vivo with a GC. A teflon container (e.g. a teflon bag) comprising a glucocorticoid and optionally a colony-stimulating factor (CSF) is also envisaged. In an even further embodiment, the present invention relates to a method of screening for a compound which is suitable for the treatment of a GC-responsive disease, said method comprising: a) contacting a monocyte with a compound to be tested; b) evaluating whether the monocyte is GC-induced; and thereby, c) identifying compounds which are suitable for the treatment of a GC-responsive disease.

Glucocorticoids (GCs) are still the most widely used immunosuppressive agents for the treatment of inflammatory disorders and autoimmune diseases. Cortisone which belongs to the group of GCs is an important therapeutic drug which is used to fight many ailments ranging from Addison's disease to rheumatoid arthritis. Ever since the discovery of its antirheumatic properties, which led to its acclaim as a wonder drug, many derivatives of cortisone with enhanced properties to better fight a specific ailment have been produced. Cortisone belongs to a group of steroids known as corticosteroids. These steroids are produced by the adrenal cortex, which is the outer part of the adrenal glands, near the kidneys. The corticosteroids are divided into two main groups: the glucocorticoids (GCs), which control fat, protein, calcium and carbohydrate metabolism, and the mineralocorticoids controlling sodium and potassium levels. Cortisone belongs to the former group, i.e. to the GCs. Cortisone and its many derivatives are used for a variety of diseases. These include endocrine disorders, rheumatic disorders, collagen diseases, dermatologic diseases, allergic states, ophthalmic diseases, respiratory diseases, hematologic disorders, neoplastic diseases, edematous diseases, gastroinstestinal diseases, etc. Specific examples include rheumatoid arthritis, tuberculosis, Addison's disease and severe asthma, to name a few. Cortisone also helped to make organ transplants a reality due to its ability to minimize the defence reaction of the body towards foreign proteins present in the implanted organ and thus damage the functionality of the implanted organ.

However, despite clinical use during more than 50 years, the specific anti-inflammatory effects of GC on different cellular compartments of the immune system are not yet clear. GCs affect nearly every cell of the immune system, and there is growing evidence for cell type-specific mechanisms.

As beneficial as GCs are as a drug, there are severe side-effects associated with their use, for example hyperglycemia due to increased gluconeogenesis, insulin resistance, and impaired glucose tolerance (“steroid diabetes”); increased skin fragility, easy bruising; reduced bone density (osteoporosis, osteonecrosis, higher fracture risk, slower fracture repair); weight gain due to increased visceral and truncal fat deposition (central obesity) and appetite stimulation; adrenal insufficiency (if used for long time and stopped suddenly without a taper); muscle breakdown (proteolysis), weakness; reduced muscle mass and repair; expansion of malar fat pads and dilation of small blood vessels in skin; anovulation, irregularity of menstrual periods; growth failure, pubertal delay; increased plasma amino acids, increased urea formation; negative nitrogen balance; excitatory effect on central nervous system (euphoria, psychosis); glaucoma due to increased cranial pressure and cataracts.

Thus, patients treated for prolonged periods of time with GCs experience deleterious side effects which limit the use of the GCs in many clinical conditions. Moreover, resistance to the therapeutic uses of glucocorticoids can present difficulty. This may be the result of genetic predisposition, ongoing exposure to the cause of the inflammation (such as allergens), and pharmacokinetic disturbances (incomplete absorption or accelerated excretion or metabolism). Due to these side effects, certain pathological conditions can not or no longer be treated with GCs.

Thus, the technical problem underlying the present invention is to provide GC-based therapies having reduced side effects.

The present invention addresses this need and thus provides, as a solution to the technical problem, a substitute GC therapy (GC-based therapy). Said substitute GC-therapy is in essence characterized by an ex vivo induction of human monocytes with one or more glucocorticoid(s).

Further embodiments of the present invention are characterized and described herein and also reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein. Unless otherwise indicated, the term “at least” preceding a series of elements is to be understood to refer to every element in the series. At least one includes for example, one, two, three, four, or five or even more.

Those skilled in the art will recognize, or be able to ascertain using no more than routine experimentation, many equivalents to the specific embodiments of the invention described herein. Such equivalents are intended to be encompassed by the present invention. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Glucocorticoid(s) (which are also denoted herein as GC or GCs (singular and plural)) are widely used for suppression of T-cell-mediated inflammations where they are considered to directly inhibit T-cells while also causing unwanted side effects. The inventors of the present invention demonstrated previously that GCs induce differentiation of an anti-inflammatory monocyte subtype (see 2). Here they reveal that these GC-induced monocytes (also denoted as “Mregs”) down-regulate antigen-specific responses of CD4+ T-cells, by suppressing proliferation and release of IFN-γ and IL-17. These effects of Mregs on T-cells require CD80 and CD124, but neither IL-10 nor Tregs (“Tregs” are regulatory T cells and represent a subset of T cells that do control other T cells by suppressing their activation. Regulatory T cells can be recognized by their surface expression of CD25 and intracellular via their expression of Foxp3 the so-called regulatory T cell-specific transcription factor). Moreover, it was demonstrated herein that the injection of Mregs (i.e. of GC-induced monocytes which have been induced ex vivo) into mice suffering from severe CD4+ T-cell-induced colitis results in dramatic clinical improvement. The present invention thus provides the first proof of concept that GC-induced monocytes (Mregs) are able to substitute a GC-therapy in a clinical setting. T-cells from spleens of treated mice also showed suppressed proliferation and secretion of IFN-γ and IL-17. Thus, GC-induced monocytes were capable to down-regulate an already established immune response in vivo. This makes Mregs a cornerstone of a novel therapeutic strategy for suppression of undesirable T-cell activation by actively and distinctly inducing resolution of inflammation in autoimmune disorders. Treatment strategies for undesirable inflammation such as allergies or autoimmune diseases are presently based on generalized suppression of inflammatory effector mechanisms. Glucocorticoids (GCs) are still the most widely used agents for suppression of both acute and chronic inflammations, but their wide spectrum of adverse effects limits long-term treatment. Their clinical efficacy in treating inflammation has been ascribed mainly to their direct inhibitory effects on activated immune cells such as T-cells. However, despite clinical use during more than 50 years, the specific anti-inflammatory effects of GC on different cellular compartments of the immune system are not yet clear. GCs affect nearly every cell of the immune system and it was thus completely unexpected that the effect of GCs on monocytes as such (ex vivo) is already sufficient to exert its beneficial effect.

Monocytes represent a central part of innate immunity. After differentiation from stem cells in the bone marrow, monocytes enter the circulation and are present in the blood until they migrate into tissues where they differentiate into macrophages or dendritic cells (DCs). These cells give rise to subtypes which are crucial for nearly every step of an immune reaction, including the initiation of an adaptive immune response, clearance of infectious agents, as well as resolution of inflammation (3-8).

Different subpopulations have been demonstrated already at the stage of circulating monocytes. Depending on their expression level of Ly6C, CX3CR1, and CCR2 murine monocytes either represent monocytes (9-11) which form tissue macrophages and dendritic cells under steady state conditions, or a “classical” phenotype which selectively migrates into inflamed tissue (reviewed in 4, 6, 12). Similarly, in the human system expression levels of CX3CR1 together with CD14, CD16 and CCR2 define comparable monocyte subtypes (6).

We have recently identified a unique population of monocytes that is induced by GCs in both human and mouse. These GC-induced monocytes present a stable and distinct phenotype (1, 2). In the murine system GCs down-regulate the expression of CCR2 and Ly6C on mouse monocytes and thus mouse GC-induced monocytes are phenotypically characterized as CX3CR1lowCCR2-Ly6Cmed/highCD80+CD124+CD163+ cells, similar to human GC-induced monocytes (1, 2).

GC-induced monocytes in humans and mice show limited adhesiveness, strong migratory and phagocytic capacity as well as production of antioxidative mediators (1, 2). Since they also show an increase in IL-10 production (1, 2), we hypothesized that GC-induced monocytes might contribute to down-regulation also of adaptive immune responses, especially those mediated by T-cells, and altogether to resolution of inflammation.

In this study we demonstrate that GC-induced monocytes indeed are effective down-regulators of CD4+ T-cell responses, and thus represent a novel phenotype we refer to as “regulatory monocytes” (Mreg). Mregs suppress CD4+ T-cell proliferation in antigen-dependent fashion, and influence their cytokine production. Much to our surprise, this down-regulation appears to be as effective as the GC treatment as such (see the appended examples). It follows that we are the first to suggest that the GC-treated human monocytes as described herein may be used as a GC-surrogate. This could not have been predicted from the art because, despite clinical use of GCs during more than 50 years, the specific anti-inflammatory effects of GC on different cellular compartments of the immune system are not clear. It was assumed, however, that GCs affect nearly every cell of the immune system. In view of this, it was unforeseeable whether the GC-induced monocytes of the present invention are already sufficient to exert the positive effects on GC-responsive diseases. The present invention is thus the first existing proof of evidence, that GCs might be used ex vivo to induce Mregs which may than be used instead of the GC itself. This will help to alleviate the severe side effects of a GC-therapy as no or just reduced amounts of GCs are to be administered to a human patient in need of a GC-therapy. Instead, one may isolate the human monocytes of the patient, induce them with GC ex vivo and administer the so-induced monocytes (Mregs) to said patient (autologous) or to an immunologically compatible patient (allogenic).

Therapeutically transfer of Mregs into mice suffering from established, severe CD4+ T-cell-induced colitis results in a suppression of proliferation and secretion of pro-inflammatory cytokine by the disease-inducing T-cells, and, most importantly, in a dramatic clinical improvement without adverse events, in particular without the side effects of a GC therapy. Thus, Mregs are valuable tools in immunotherapy of inflammatory disorders that could substitute for conventional systemic GC therapy without displaying their severe side effects. Such an induction of active resolution of T-cell mediated disease appears to be a general principle for novel therapeutic strategies to suppress overwhelming inflammatory processes.

The present invention, thus relates in a first embodiment to a composition, preferably a pharmaceutical composition, comprising glucocorticoid (GC)-induced human monocytes (Mregs), and optionally a pharmaceutically acceptable carrier and/or diluents. Said pharmaceutical composition is preferably for use in the treatment of a disease which is GC-responsive.

The present invention further relates to a pharmaceutical composition comprising at least one GC and optionally a pharmaceutically acceptable carrier, for use in the treatment of a disease which is GC-responsive, wherein said pharmaceutical composition is to be administered to human monocytes ex vivo such that said human monocytes become (are) GC-induced.

Said pharmaceutical composition is thus for the ex vivo administration. The present invention thus relates in a further embodiment to a pharmaceutical composition comprising at least one GC and optionally a pharmaceutically acceptable carrier/diluent which composition is used for the treatment, amelioration or prevention of a GC-responsive disease, characterized in that said composition is to be administered to human monocytes (or blood based products comprising human monocytes) of the human patient ex vivo, wherein said patient suffers or is assumed to suffer from said GC-responsive disease, and wherein the GC-treated human monocytes are to be administered to said human patient.

In another embodiment, the present invention relates to a method for the preparation of a composition, preferably a pharmaceutical composition, comprising the step of contacting human monocytes ex vivo with at least one GC. Said contacting is to be carried out such that the human monocytes become/are GC-induced.

The present invention also relates to the use of at least one GC for the preparation of a pharmaceutical composition for the treatment of a patient in need of a GC-therapy, wherein said GC is to be administered to monocytes of said patient ex vivo such that said monocytes become (are) GC-induced. “A patient in need of a GC-therapy” is a human patient who suffers from and/or shows the symptoms of a GC-responsive disease. The latter term is explained herein elsewhere.

It is also envisaged that these GC-induced human monocytes are (to be) administered subsequently (i.e. after the ex vivo induction with at least one GC) to that patient. It is thereby envisaged that the so-induced monocytes (Mregs) are (to be) administered to the patient from which they have been isolated before (autologous) or to an immunologically compatible patient (allogenic). The pharmaceutical compositions, uses and methods of the present invention are preferably used in an autologous or an allogeneic setting.

It is further envisaged that the GC-induced human monocytes are further treated before they are to be (re)administered to that patient. “Further treated” includes (a) cell culture measures to keep or store the GC-induced monocytes for a desired period of time (particularly up to the time point where these cells are (to be) (re)administered); (b) measures to wash the GC-induced monocytes in order to dilute or even remove residual amounts of the GC which was used for the induction (“dilute” preferably to such an extent that the GC is in a concentration which equates with or falls below the GC-concentration which exerts or is assumed to exert side-effects in a human); (c) measures to (further) enrich the monocyte population; (d) measures to enrich the GC-induced monocytes for at least one specific cell marker which is or is assumed to be characteristic for the GC-induction (these markers are specified in great detail herein); and/or (e) methods to further induce said GC-induced monocytes, for example by co-administering M-CSF, GM-CSF, Interleukin-4 (IL-4), or IL-10 etc.

The term “ex vivo”, which is interchangeable with “in vitro”, refers to activities conducted in a controlled environment which is apart from the human body. As used herein and in the art, this term is often used interchangeably with “in culture”.

The terms “glucocorticoid (GC)-induced human monocytes”, “GC-induced regulatory monocytes” or “Mregs” are interchangeable. All these terms refer to a population of human monocytes which have been induced ex vivo with a glucocorticoid or GC and/or pharmaceutically acceptable derivatives thereof. An exemplary protocol for the GC-induction of monocytes is shown in Example 2.

Monocytes represent a central part of innate immunity. After differentiation from stem cells in the bone marrow, monocytes enter the circulation and are present in the blood until they migrate into tissues where they differentiate into macrophages or dendritic cells (DCs). Different subpopulations have been demonstrated already at the stage of circulating monocytes. Depending on their expression level of Ly6C, CX3CR1, and CCR2 murine monocytes either represent monocytes (9-11) which form tissue macrophages and dendritic cells under steady state conditions, or a “classical” phenotype which selectively migrates into inflamed tissue (reviewed in 4, 6, 12). Similarly, in the human system expression levels of CX3CR1 together with CD14, CD16 and CCR2 define comparable monocyte subtypes (6).

There are two types of monocytes in human blood: (a) the classical monocyte, which is characterized by high level expression of the CD14 cell surface receptor (CD14++ monocyte) and (b) the non-classical, pro-inflammatory monocyte with low level expression of CD14 and with additional co-expression of the CD16 receptor (CD14+CD16+ monocyte). Both subtypes express CD14 on the cell surface and it is to be understood that a reference to a “CD14 monocyte” or to a “human monocyte” includes both of the above subtypes. It is thus envisaged that both subtypes of human monocytes are within the scope of the present invention, the CD14++ subtype being preferred.

Methods to isolate such CD14 positive monocytes are well-known in the art and are also described in sufficient detail herein elsewhere (e.g. in the appended example, see in particular Example 2). It is therefore within the scope of the present invention to isolate CD14 positive monocytes as set out in the examples (for example as set out in Example 2). To this end, one may use a suitable gradient centrifugation step, for example a Ficoll gradient centrifugation, followed by a CD11c+, CD19+ and CD90+ cell depletion, e.g. by way of magnetic cell sorting MACS.

It is in any way within the common general knowledge of a skilled person to enrich human CD14 positive monocytes, for example by way of FACS aided cell-sorting or by way of established isolation facilities like, for example, the CliniMACS Cell Separation System from Miltenyi Biotec.

The “starting material” for the isolation of CD14 positive monocytes is not further limited and includes blood (or whole blood), the separation product of a whole blood leukapheresis, bone marrow or other blood based starting materials comprising human monocytes such as cord blood or PBMCs, as well as pleural, peritoneal, or synovial fluids or from various tissues, such as spleen and lymph node. Leukapheresis is a laboratory procedure with which white blood cells (leucocytes) are separated from a sample of blood. The leukapheresis procedure is well known to the skilled person.

In a further embodiment of the present invention, the CD14 positive human monocytes are isolated/manufactured by way of a method comprising
a) Optionally providing the starting material for the isolation of CD14 positive monocytes (e.g. providing a suitable container such as a vial which contains the starting material for the isolation);
(b) Optionally washing the cells in order to remove and lyse erythrocytes (for example with a Cell washer);
(c) Labelling of the cells with CD14 specific binding domain (for example CD14 binding beads (Miltenyi Biotech or others);
(d) Separating CD14 positive cells, preferably based on a CD14 specific binding agent (for example by way of cell sorting for example with a CliniMACS cell sorting system (Miltenyi Biotech).

Said method may further comprise the step of:
(e) Seed monocytes in a cell culture device (for example a Teflon bag)
(f) Bringing the cells into contact with at least one GC (preferably for 48 hours) and optionally M-CSF (preferably for 48 hours);
(g) Optionally washing the cells; and
(h) Optionally providing the cells in a pharmaceutically acceptable form (i.e. in a pharmaceutically acceptable carrier or diluents).

A “CD14 specific binding domain” characterizes in connection with the present invention a domain of a polypeptide which specifically binds to/interacts with CD14. Said binding/interaction is also understood to define a “specific recognition of CD14”. The term “specifically recognizing CD14” or “specific for CD14”, means in accordance with the present invention that the binding domain, e.g. an antibody, is capable of specifically interacting with and/or binding to human CD14. As used herein, the term “binds” in connection with the interaction between CD14 and a binding domain indicates that the binding domain associates with (e.g., interacts with or complexes with) CD14 to a statistically significant degree as compared to association with proteins generally (i.e., non-specific binding). Thus, the term “binding domain” is also understood to refer to a domain that has a statistically significant association or binding with CD14. The CD14 specific binding domain is preferably labelled, e.g. with a fluorescent label or beads (such as magneto beads) which allow a separation of CD14 positive cells in a cell sorting device.

The binding domain of the present invention preferably is or comprises an epitope binding domain. Preferably, said epitope binding domain is an antibody or an antigen binding fragment thereof. The term “antibody” refers to a monoclonal or a polyclonal antibody (see Harlow and Lane, “Antibodies, A Laboratory Manual”, CSH Press, Cold Spring Harbor, USA, 1988) which binds to a target, or a derivative of said antibody which retains or essentially retains its binding specificity. Preferred derivatives of such antibodies are chimeric antibodies comprising, for example, a mouse or rat variable region and a human constant region. The term “antibody” also comprises bifunctional (bispecific) antibodies and antibody constructs, like single-chain Fvs (scFv) or antibody-fusion proteins. The term “scFv fragment” (single-chain Fv fragment) is well understood in the art and preferred due to its small size and the possibility to produce such fragments recombinantly. Said antibody or antibody binding portion is a human antibody or a humanized antibody. The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO 90/07861. The term antibody or functional fragment thereof also includes heavy chain antibodies and the variable domains thereof, which are mentioned in WO 94/04678, WO 96/34103 and WO 97/49805, WO 04/062551, WO 04/041863, WO 04/041865, WO 04/041862 and WO 04/041867; as well as domain antibodies or “dAb's”, which are based on or derived from the heavy chain variable domain (VH) or the light chain variable domain (VL) of traditional 4 chain antibody molecules (see, e.g., Ward et al. 1989 Nature 341, 544-546).

The term “antigen binding fragment” as used herein refers to fragments of the antibodies as specified herein which retain or essentially retain the binding specificity of the antibodies like, separated light and heavy chains, Fab, Fab/c, Fv, Fab′, F(ab′)2. An antigen-binding fragment may comprise a light chain variable region (VL) and a heavy chain variable region (VR) of an antibody; however, it does not have to comprise both. Fab fragments, for example, have two VH regions and often retain antigen-binding function of the intact antigen-binding fragment.

The term “epitope binding domain” includes, besides antibodies or functional fragments thereof, other binding entities which bind to (specifically bind to) a target. The term “epitope binding domain” includes, for example, a domain that (specifically) binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin (adnectin); which has been subjected to protein engineering in order to obtain binding to a ligand other than the natural ligand. CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001).

A preferred example of a binding domain in line with the present invention is an antibody, more preferably a monoclonal antibody.

Said isolation of CD14+ monocytes may be carried out according to the clinical procedure developed by Miltenyi Biotech for the isolation of CD14 positive cells from a leukapheresis product of whole blood. Said method makes use of the CliniMACS system (Miltenyi Biotech). It is envisaged that the CD14 positive cells are isolated by way of the respective cell isolation protocol published by Miltenyi Biotech in respect of the CliniMACS or MACS cell separation system.

A suitable “starting material” for the GC-induction of human monocytes is, for example, any fluid/buffer comprising CD14 positive human monocytes, or a blood based product comprising CD14 positive human monocytes which blood based product, fluid and/or buffer is optionally depleted of CD11c+, CD19+ and CD3+ cells. Monocytes which are (or which are to be) contacted with at least one GC include monocytes which are comprised in the starting material for isolation of monocytes or in the starting material for the GC-induction.

The term “depleted of CD11c+, CD19+ and CD3+ cells”, however, does not exclude that portions, preferably small portions, of these cells are still present. Small portions in this regard includes that up to about 25, 20, 15, 10, 5, 1, 0,5 or 0.1 (or even less)% of the total cell population, consist of CD11c+, CD19+ and CD3+ cells, e.g. those which were isolated together with the envisaged CD14 positive monocytes. To the more, it is not excluded that the CD14 positive monocyte population contains a 25, 20, 15, 10, 5, 1, 0,5 or 0.1 (or even less)% contamination with other cell types (other than the above mentioned CD11c+, CD19+ and CD3+ cells, for example with NK cells and NKT cells to name some). The quality of the CD14 positive monocytes may be observed by standard methods.

It is preferred that at least 50% of the total cells which are used for the subsequent GC-induction are CD14 positive human monocytes, more preferably at least 75, 80, 85% and even more preferred at least about 90% or even more (up to 100%) of the total cell number which are used for the GC-induction are CD14 positive human monocytes. A “blood based product” includes natural or artificial products which are based upon human whole blood, including whole blood

It is particularly preferred that the CD14 positive monocytes as mentioned herein (i.e. those which are to be stimulated with GCs) are of human origin.

CD14 positive human monocyte cells which were isolated by way of the respective CliniMACS Cell Separation System (Miltenyi Biotec) from a whole blood leukapheresis separation product are particularly preferred. Said procedure results in a CD14 positive monocyte suspension which contains 10% or less impurity by other cells, i.e. the CD14 positive monocytes have a degree of purity of about 90, 95, 97,5% or even 100% (CD14 positive monocytes/total cell number).

The induction of the above-mentioned monocytes with the glucocorticoid or GC and/or pharmaceutically acceptable derivatives thereof is to be carried out such that at least about 25, preferably about 50% and more preferably about 95% or more of the total cell number of the GC-induced CD14-positive human monocytes is characterized by at least by one, preferably by all, of the following characteristics:
(1) CD163 positive on mRNA and/or protein level, preferably at the protein level—it is even more preferred that about 80% or more of the CD14-positive human monocytes are CD163 positive on protein level
(2) CD121b positive on mRNA and/or protein level, preferably at the protein level—it is even more preferred that the CD14-positive human monocytes show an increase of CD121b by at least 10% on protein level (CD121b is also known as the Interleukin 1 receptor, type II (IL1R2));
(3) CD11b positive on mRNA and/or protein level, preferably at the protein level —it is even more preferred that about 80% or more of the CD14-positive human monocytes are CD11b positive on protein level
(4) Induction of CD80 mRNA and/or increased (relative to the non-GC-induced cells) expression of CD80 protein on the cell surface by at least 5%
(5) Upregulation (relative to the non-GC-induced cells) of the IL4-receptor alpha chain (CD124) mRNA and/or on protein level by at least 5%

It is also envisaged that the at least about 25, preferably about 50% and more preferably about 95% or more of the total cell number of the GC-induced CD14-positive human monocytes is additionally characterized by at least by one of the following characteristics:
(6) CX3CR1^{low}—down-regulation (mRNA and/or protein)—preferably at the protein level by at least 5%
(7) IL10 positive—upregulation (mRNA); at least 3-fold
(8) CD38 positive—upregulation (mRNA and/or protein); preferably at the protein level by at least 5%
(9) CCR2low—down-regulation (protein and/or mRNA) preferably at the protein level by at least 5%

“At least about 25%” means that at least about 25%, i.e. 25, 30, 35, 40, 45, 50, 60, 70, 89, 90 or even 99, or 100% of the total cell number of GC-induced CD14-positive human monocytes is characterized, by way of the GC-stimulation, by at least one, i.e. one, two, three, four, five, six, seven, eight and/or nine (all) of the above characteristics (1) to (9).

In a preferred embodiment, it is envisaged that at least about 25% of the total cell number of the GC-induced CD14-positive human monocytes is at least characterized by the above identified characteristics (1) to (5) relating to CD163, CD11b, CD121b, CD80 and CD124 (preferably in regard to the protein level).

It is however also envisaged that at least about 25% of the total cell number of the GC-induced CD14-positive human monocytes is at least characterized by the above identified characteristics (1), (2), and (7) relating to CD163, CD121b and IL-10 in regard to the mRNA level. Means and methods to measure such mRNA levels are well known to the skilled person.

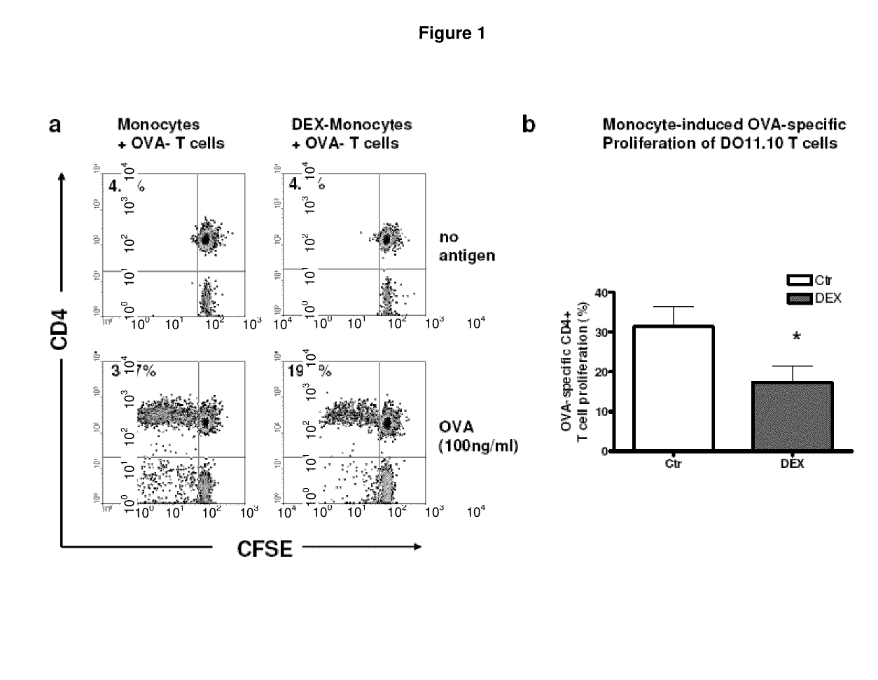

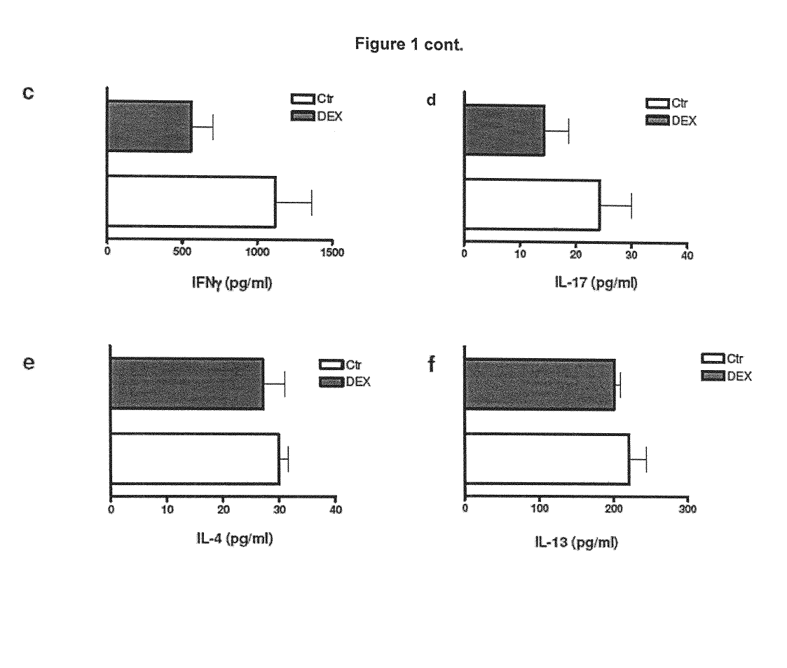

The Mregs of the present invention can be further characterized in that they are able to reduce CD4+ T-cell proliferation in antigen-dependent fashion, by at least 10% preferably at least 25, 30, 35, 40, 50%, or even more. We used OVA TCR transgenic T-cells from DO11.10 mice as cells responding to OVA peptide presented by monocytes. We prepared splenic CD4⁺ T-cells and performed co-cultures with monocytes purified from bone marrow with and without OVA peptide. When OVA-peptide was presented by monocytes (FIG. 1a), OVA-specific CD4⁺ T-cells revealed considerable proliferation (34.7%). However, when Mregs were used to present OVA, CD4⁺ T-cell proliferation was remarkably and significantly reduced (FIG. 1b).

When we measured the effects on cytokine production (per cell), we found that control antigen-presenting monocytes induced release of IL-4 (FIG. 1e) and IL-13 (FIG. 10 as well as IFN (FIG. 1c) and IL-17 (FIG. 1d). However, when pre-treated with DEX, antigen-presenting Mregs selectively induced release of IL-4 (FIG. 1e) and IL-13 (FIG. 1f) in similar amounts, while production of IFN (FIG. 1c) and IL-17 (FIG. 1d) was suppressed.

Human Mregs of the present invention are alternatively or additionally characterized in that they are able to (a) elicit Ag-specific responses in T-cells and/or (b) to inhibit proliferation by at least 10% and pro-inflammatory cytokine response by at least 10% (IFN and IL-17) compared to control monocytes which have not been induced with a GC, for example in a setting as described above together with the description of FIG. 1.

Genome wide expression screening of dexamethasone-(DEX)-treated human monocytes revealed the induction of an anti-inflammatory phenotype (1). Phenotypically and by functional assays we recently described a similar murine subset induced by GC treatment (2). We now performed genome wide expression screening of these murine GC-induced monocytes. Functional clustering based on overrepresentation of gene ontology annotations among regulated genes (1, 13) demonstrated that GC treatment altered the capacity of monocytes to interact with lymphocytes and thus cells of the adaptive immunity (Supplemental Table 1 and 2).

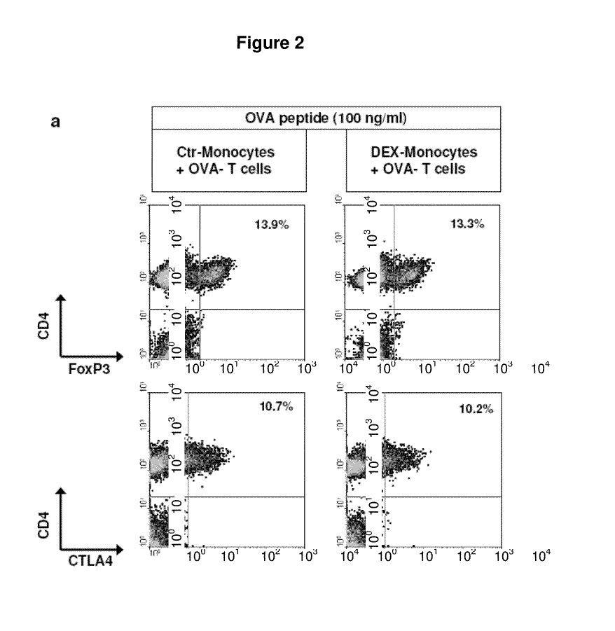

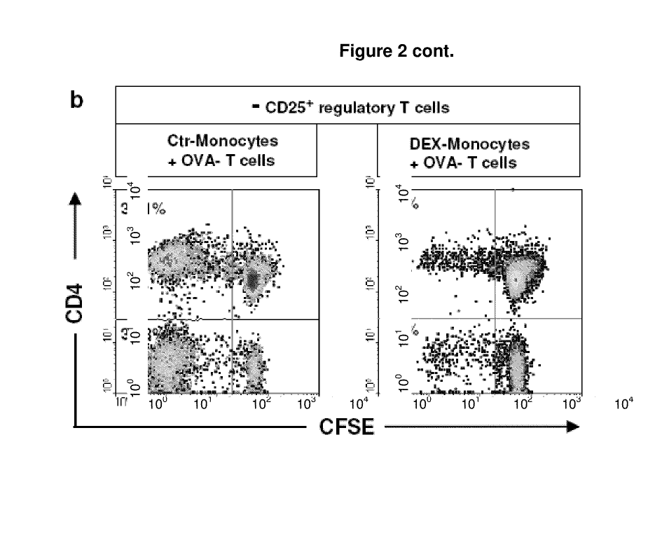

We next explored mechanisms that are used by Mregs to regulate CD4⁺ T-cell activation. One possible way would be through induction of Tregs, as shown for IFN-treated macrophages (14). In order to test whether Tregs are induced by Mregs we performed co-cultures of CD4⁺ T-cells and OVA-pulsed Mregs and after 7 days analyzed expression of Foxp3 and CTLA-4 (CD152), the major signatures of Tregs (15) in CD4⁺ T-cells. The percentage of Foxp3⁺CD4⁺ T-cells and that of CTLA-4⁺CD4⁺ T-cells did not differ whether Mregs or Ctr-Mo presented antigen (FIG. 2a). To exclude the possibility that Mregs induce functional Tregs we removed CD25⁺ cells by magnetic beads prior to the co-culture of CD4⁺ T-cells with either Ctr-Mo or Mregs. As shown in FIG. 2b, overall T-cell proliferation was enhanced when Tregs (CD25⁺ T-cells) were removed, while the presence of Mregs still resulted in efficient down-regulation of T-cell proliferation (FIG. 2b). These data show that Mregs confer their regulatory function directly to CD4⁺ effector T-cells, and not via Tregs.

Human Mregs of the present invention are alternatively or additionally characterized in that they do not induce Tregs, e.g. in a setting as described above together with the description of FIG. 2 (no statistically significant induction of Tregs).

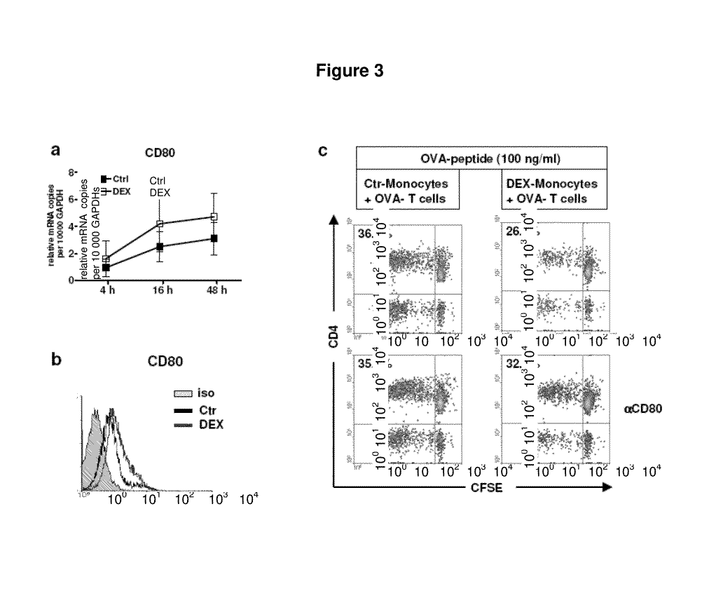

After treatment with GC, Mregs showed induction of CD80 on the mRNA level (FIG. 3a) and increased expression of CD80 protein on the cell surface (FIG. 3b). This generally costimulatory molecule turns inhibitory towards T-cells when it interacts with Programmed death-ligand 1(PD-L1) (16). PD-L1 was present on CD4⁺ T-cells, though its expression was not increased on cells co-cultured with Mregs instead of Ctr-Mo (data not shown). In order to test then whether the upregulation of CD80 on Mregs is of functional relevance for regulation of T-cells, we added neutralizing CD80 antibody during co-culture of OVA-pulsed Mregs or Ctr-Mo with OVA-specific CD4⁺ T-cells (FIG. 3c). The inhibition of the interaction between CD80 and its ligand distinctly abolished the suppressive effects of Mregs on CD4⁺ T-cell proliferation (FIG. 3c), indicating that CD80 mediates Mregs-induced regulation of T-cell proliferation.

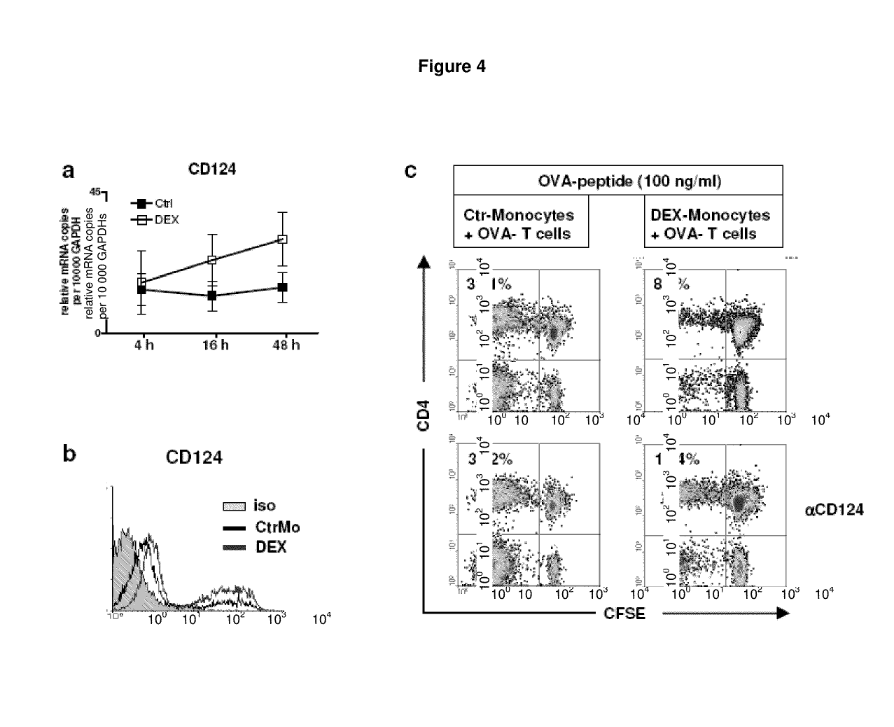

We have recently reported that some facets of Mregs resemble the so called “myeloid-derived suppressor cells” (MDSC), which have been originally discovered in tumor bearing mice and humans (17, 18). One important parallel is the upregulation of the IL-4 receptor-alpha chain (CD124) (FIGS. 4a and 4b) (2) through which MDSC partly confer their regulatory function on T-cells (19). To test whether CD124 is similarly relevant for the inhibitory effect of Mregs on CD4⁺ T-cells we used anti-CD124 antibody during presentation of OVA by Mregs to OVA TCR-tg CD4⁺ T-cells (FIG. 4c). The antibody had no effect on proliferation of T-cells induced by Ctr-Mo, but it significantly inhibited the ability of Mregs to keep down T-cell proliferation (FIG. 4). Thus, IL4R and CD80 both mediate mechanisms displayed by Mregs in T-cell regulation.

Since IL-10 mRNA, a classical immunosuppressive cytokine, was upregulated in Mregs upon DEX treatment (2) we employed a blocking anti-IL-10 antibody in our OVA assay to assess whether released IL-10 is involved in the down-regulatory effects of Mregs on T-cell proliferation. We did not detect any influence of the anti-IL-10 antibody with respect to Mreg-induced T-cell proliferation (Supplemental FIG. 1) indicating that CD4⁺ T-cell regulation by Mregs is not dependent on IL-10 although IL-10 mRNA is significantly upregulated on Mreg by at least 2-fold when compared to Ctr.-monocytes.

We next analyzed whether Mregs exert similar effects on CD4⁺ T-cells in vivo and thus mediate actions of GC in situ. We therefore investigated their effects in CD4-dependent inflammatory bowel disease (IBD) (20-22). In this model, syngeneic CD4⁺CD25⁻ T-cells induce severe and fatal colitis when adoptively transferred into Rag^{-/-} mice (21). Colitis is monitored clinically by weight loss.

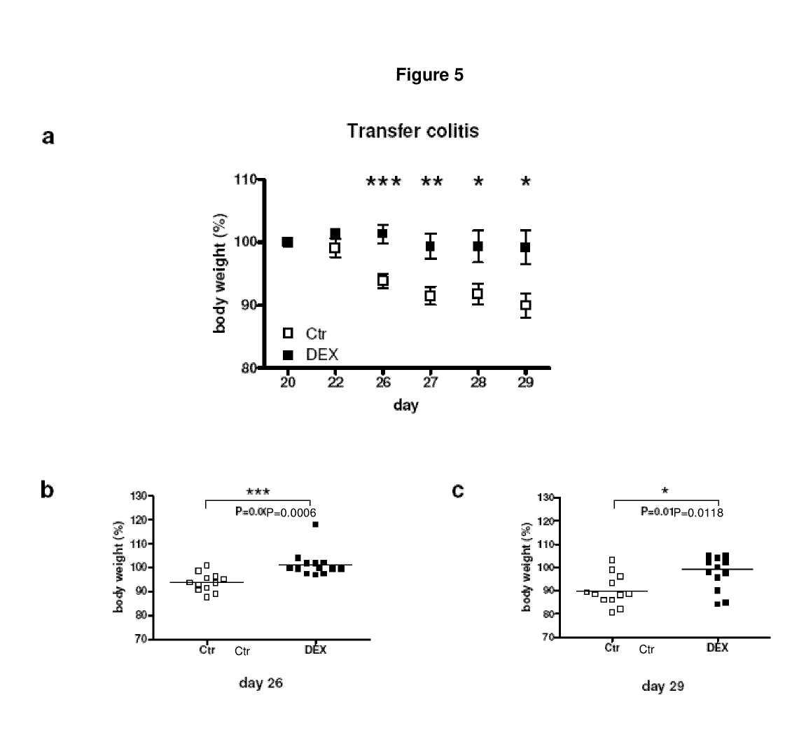

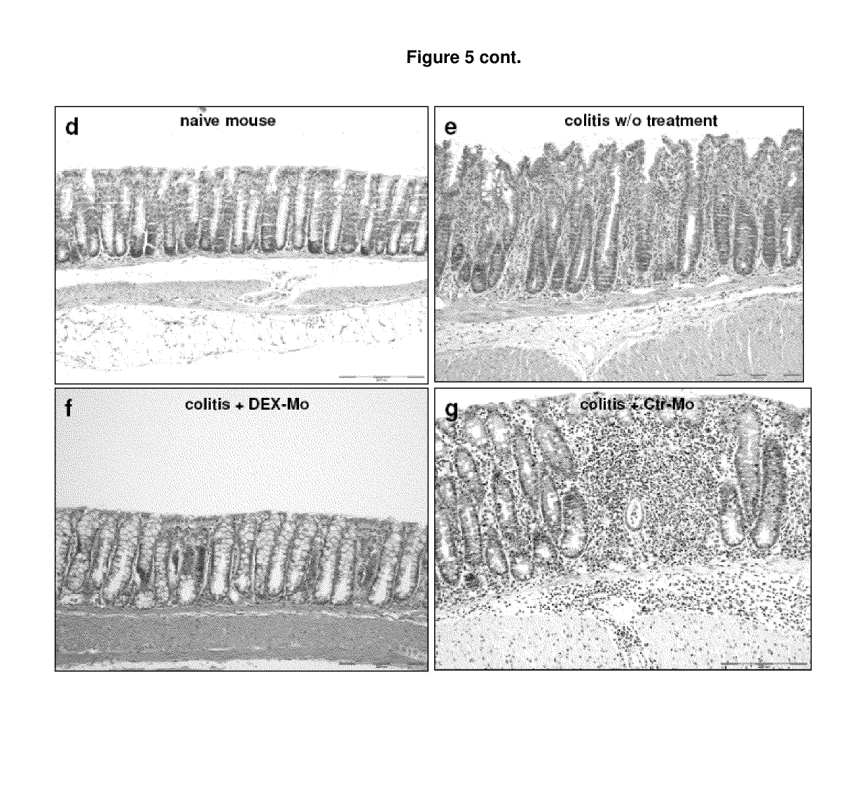

We injected either vehicle, Ctr-Mo or Mregs i.v. after mice had developed severe colitis, indicated by lost weight on consecutive days (approx. 3 weeks after eliciting colitis by injection of CD4⁺CD25⁻ T-cells). Mice that had received Mregs showed significant clinical improvement over a period of 9 days post cell transfer (FIG. 5a). In contrast, mice that had received Ctr-Mo continued to lose weight and had to be sacrificed on day 29 (FIG. 5a-c), identically to mice which received vehicle only (data not shown). In those experiments where mice with colitis were monitored for longer periods post Mreg-transfer, 3 out of 4 animals that had received Mregs survived, while 3 out of 4 mice that were untreated died by day 60 (Supplemental FIG. 2). When colons were removed from sacrificed mice at day 29 and evaluated histologically, we observed large inflammatory infiltrates in untreated colitis mice (FIG. 5e) and in mice that received Ctr-Mo (FIG. 5g). However, these infiltrates had almost completely resolved after injection of Mregs (FIG. 5f, compare to sections of naïve mice FIG. 5d).

Taken together, treatment of already established, severe CD4⁺ T-cell-induced colitis by injection of Mregs led to complete resolution of inflammation. Thus, Mregs not only selectively regulate Ag-specific responses in T-cells while presenting antigen in vitro, but they exert regulatory effects on activated CD4⁺ T-cell also in vivo.

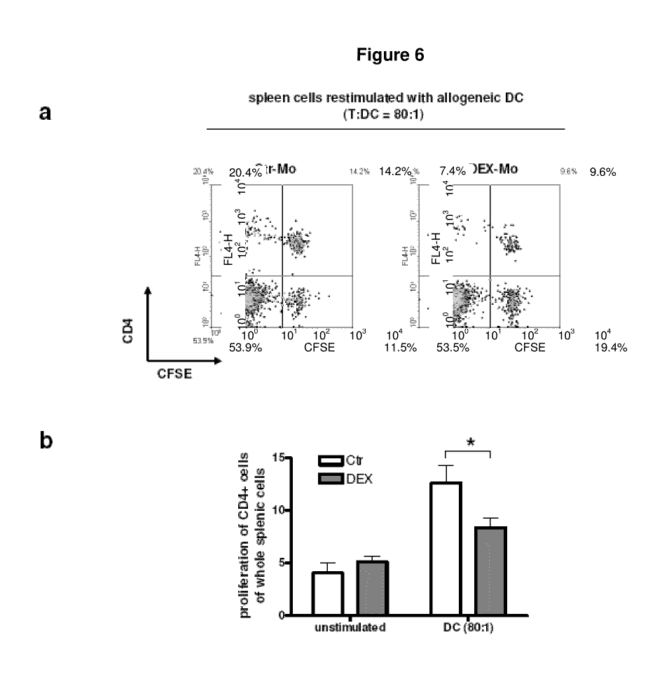

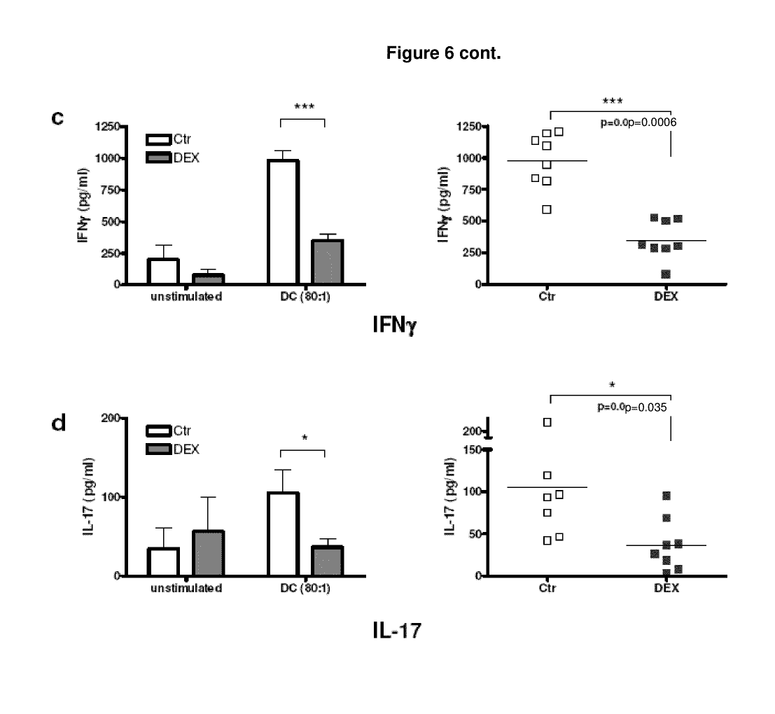

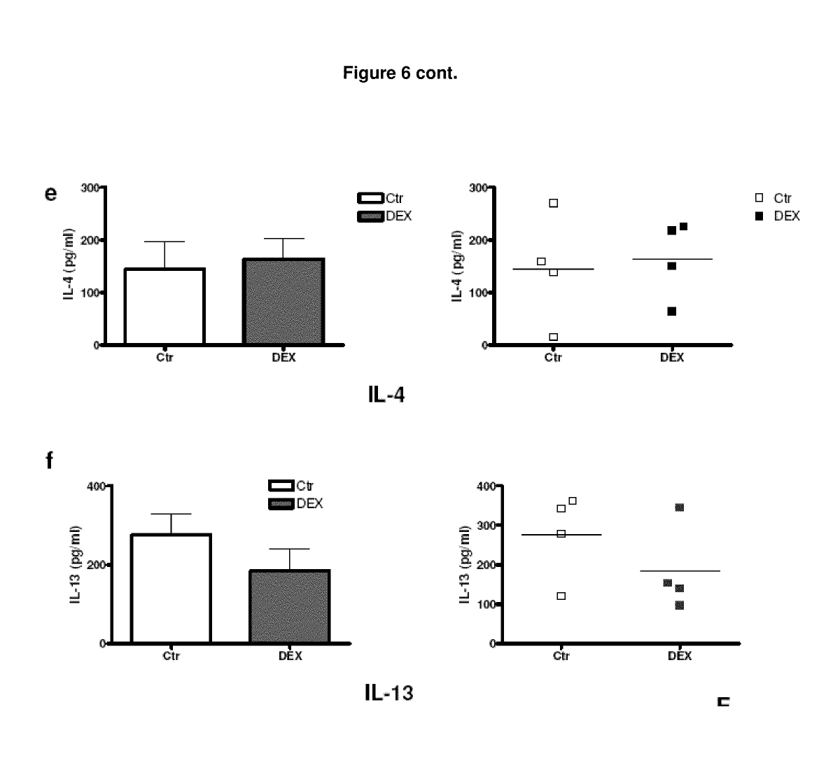

To demonstrate that resolution of inflammation in colitis by Mregs was indeed due to regulation of activated CD4⁺ T-cells, we examined splenic T-cells from mice with colitis on day 29. Since Rag^{-/-} mice do not have intrinsic T-cells, we first ascertained the presence of CD4⁺ T-cells in the spleens adoptively transferred 30 days before (data not shown and FIG. 6a). We then labeled whole spleen cells with CFSE and restimulated these with allogeneic DC in order to test their capacity to be activated and to proliferate. Analysis after 5 days of co-culture showed that DC did indeed induce CD4⁺ T-cell proliferation (FIG. 6a). However, CD4⁺ T-cells isolated from those mice which had been therapeutically injected with Mregs showed markedly reduced proliferation compared to mice that had received Ctr-Mo (FIGS. 6a and 6b). So Mregs had down-regulated CD4⁺ T-cell activation in vivo to an extent which was still detectable by re-stimulation ex vivo (FIG. 6b).

When we measured cytokine production of CD4⁺ T-cells in transfer colitis we found that splenic T-cells from mice having received Mregs produced similar amounts of IL-4 (FIG. 6e) and IL-13 (FIG. 6f) compared to T-cells from mice treated with Ctr-Mo. In contrast, IL-17 and especially IFN production was significantly suppressed in CD4⁺ T-cells from mice treated with Mregs (FIG. 6c, d).

Thus, therapeutic transfer of Mregs in vivo resulted in an alteration of cytokine pattern of stimulated T-cells which was still present when these T-cells were re-stimulated ex vivo with allogeneic DC.

The term “glucocorticoid or GC and/or pharmaceutically acceptable derivatives thereof” includes substances that bind, preferably specifically, to the glucocorticoid receptor. Said term includes particularly at least one, i.e. one, two, three, four, five or even more compound(s) selected from the group consisting of cortisone, cortisol (hydrocortisone), cloprednol, prednisone, prednisolone, methylprednisolone, deflazacort, fluocortolone, triamcinolone, dexamethasone, beatamethasone, cortivazol, paramethasone, and/or fluticasone, including pharmaceutically acceptable derivatives thereof. The mentioned compounds may be used alone or in any combination.

It will be readily appreciated that the present invention is not limited to the above mentioned specific GCs as it is envisaged that all substances which already are or will be classified as a “glucocorticoid”, may be employed in the context of the present invention. Such future glucocorticoids include compounds which specifically bind to and activate the glucocorticoid receptor. The term “specifically binds to the GC receptor” means in accordance with the present invention that the GC (or a compound which is assumed to act like a GC) associates with (e.g., interacts with) the GC receptor (also known as NR3C1) to a statistically significant degree as compared to association with proteins/receptors generally (i.e., non-specific binding). When the GC receptor binds to glucocorticoids, its primary mechanism of action is the regulation of gene transcription. In the absence of GC, the glucocorticoid receptor (GR) resides in the cytosol complexed with a variety of proteins including heat shock protein 90 (hsp90), the heat shock protein 70 (hsp70) and the protein FKBP52 (FK506-binding protein 52). The binding of the GC to the glucocorticoid receptor (GR) results in release of the heat shock proteins.

It is thus envisaged that a future GC, or a pharmaceutically acceptable derivative or salt of a GC is preferably able to bind to the GC receptor and to release the above mentioned heat shock proteins. After the receptor is bound to glucocorticoid, the receptor-glucocorticoid complex can take either of two paths.

The activated GR complex up-regulates the expression of anti-inflammatory proteins in the nucleus or represses the expression of pro-inflammatory proteins in the cytosol (by preventing the translocation of other transcription factors from the cytosol into the nucleus). It is also envisaged that a future glucocorticoid is a substance which mimics the action of a GC, and which is still able to induce human CD14-positive monocytes as described above. Preferably, said future GC has a comparable biological function when compared with dexamethasone. “Comparable biological function” means that the derivatives of the invention are still able to act as a inducer of human CD14-positive monocytes with a deviation of the inducing activity in respect to dexamethasone, of not more than about 40%, 30%, 20%, 15%, 10%, 5%, 2,5%, 2% or 1%, for example under conditions which equate to or are identical with those set out in Example 2.

In a preferred embodiment, said GC is selected from the most clinical used and relevant GCs like dexamethasione, fluticasonepropionate, prednisolone, methylprednisolone, betamethasone, triamcinolonacetonide or combinations thereof.

In an even more preferred embodiment, said GC is dexamethasone.

The term “pharmaceutically acceptable derivatives” includes salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatisation. It is envisaged that these derivatives are still capable to induce human CD14-positive monocytes as described above. Preferably, said derivatives have a comparable biological function when compared with dexamethasone. “Comparable biological function” means that the derivatives of the invention are still able to act as a inducer of human CD14-positive monocytes with a deviation of the inducing activity in respect to dexamethasone, of not more than about 40%, 30%, 20%, 15%, 10%, 5%, 2,5%, 2% or 1%, for example under conditions which equate to or are identical with those set out in Example 2.

The term “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolarnine, tromethamine, N-methylglucamine, and the like.

Examples of suitable pharmaceutically acceptable carriers and/or diluents are well known in the art and include phosphate buffered saline solutions, water, emulsions, such as oil/water emulsions, various types of wetting agents, sterile solutions etc. Compositions comprising such carriers can be formulated by well known conventional methods. It is for example envisaged to store and/or to administer the cells of the present invention in serum free media which are typically used for the cell processing and strorage of DC (dendritic cells) cells. Exmaples of such media are well known and include by way of example the CellGro® DC media of CellGenix.

It is also envisaged that the Mregs are suspended and/or diluted in cell culture buffers like PBS or, medium (cell culture medium) plasma, serum, whole blood or any other medium.

These pharmaceutical compositions of the present invention can be administered to a subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 2−10×10⁶Mregs/patient; however, doses below or above this exemplary range are envisioned, especially considering the aforementioned factors. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use.

The oral and/or topical administration of the pharmaceutical compositions of the invention is less preferred.

Upon using the compositions of the present invention, it is possible to treat/ameliorate and/or prevent diseases or medical conditions which are GC-responsive. It is thus envisaged that the Mregs of the present invention are used for the preparation of a pharmaceutical composition for the treatment of diseases or medical conditions which are GC-responsive. For an overview see: Rhen, T. and Cidlowski, J. A. (2005). Antiinflammatory action of glucocorticoids—new mechanisms for old drugs. N. Engl. J. Med. 353, 1711-1723.

and Kaiser, Kley: Cortisontherapie, 11. Auflage, Thieme, 2002.

The term “GC responsive” includes diseases and/or medical conditions which can be treated, ameliorated and/or prevented or which are assumed to be treatable, amelioratable and/or preventable by a GC. The capability to treat, ameliorate or prevent a disease by way of a GC-treatment is either already known for that disease/medical condition or will turn out in the future.

A “GC responsive” disease is preferably a disease which falls in the category of internal medicine. Examples of such diseases include inflammatory diseases, autoimmune diseases, endocrine disorders, rheumatic disorders, collagen diseases, dermatologic diseases, allergic states, ophthalmic diseases, respiratory diseases, hematologic disorders, neoplastic diseases, edematous diseases, gastroinstestinal diseases, etc. to name a few.

The GC-responsive diseases which are to be treated, ameliorated and/or prevented within the context of the present invention are preferably T-cell mediated diseases, more preferably CD4+ positive T-cell mediated diseases. A “T cell mediated disease” is a disease for which it is known or for which it will be known or for which it is assumed that T-cells, preferably CD4 positive T-cells, do exert a negative effect which negative effect is either causative for that disease and/or is a concomitant effect (at least in part) which at least jointly responsible for that disease. A “negative effect” includes all kinds of unwanted effects that a T-cell is known to exert, or is assumed to exert in the body of a subject, preferably a human subject. Examples of such negative effects that a T-cell might exert are well-known and can be exemplified for example autoreactive T cells that do recognize body own structures like DNA, Islet cells, myelin basic protein and destroy them like in the below listed diseases.

Specific examples of a T-cell mediated disease include, but are not limited to, the following diseases: Atopic Dermatitis; Asthma; Colitis Ulcerosa; Morbus Crohn; Psoriasis vulgaris and Psoriatic arthritis; Autoimmune Skin Disorders (Phemphigus, Pemphigoid); Multiple Sklerosis; Rheumatoid Arthritis; Type I diabetes (IDDM) (Tisch-R, and Wang-B, Adv. Immunol.: 100: 125-49, 2008); Systemic Lupus Erythematodes; Dermatomyositis, Polymyositis; Graft-versus-host disease (Hess-AD Biology of Blood and Marrow Transplantation: 12: 13-21. 2006), to name some.

It is preferred that the medical conditions/disorders to be treated, ameliorated and/or prevented within the context of the present invention are chronic diseases. A “chronic disease” is a disease that is long-lasting or recurrent. A skilled practitioner in the medical field can decide without further ado whether a disease is chronic or not. This depends of course on the specific disease, the time the disease already lasted etc.

The terms “treatment”, “treating” and the like are used herein to generally mean obtaining a desired pharmacological and/or physiological effect. The effect may be prophylactic in terms of completely or partially preventing a GC-responsive disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response.

In a further aspect, the present invention relates to a (pharmaceutical) kit or pharmaceutical package comprising at least one GC and optionally means to induce CD14 positive monocytes ex vivo, and/or means to enrich CD14 positive monocytes, and/or means to purify or enrich GC-induced monocytes; and/or means to wash GC-induced monocytes; and/or means to store GC-induced monocytes or monocytes which have not yet been induced by a GC. Said GC and the additional means are thereby preferably packaged together in one sealed pharmaceutical package or kit. Parts of the kit and package of the invention can be packaged individually in vials or bottles or in combination in containers or multicontainer units. The manufacture of the kits follows preferably standard procedures which are known to the person skilled in the art.

The pharmaceutical package or kit may also comprise written instructions for the GC-induction of monocytes in accordance with the methods of the present invention. Said pharmaceutical package or kit may further comprise a label or imprint indicating that the contents can be used for the GC-induction of CD14 positive monocytes, and/or for treating, ameliorating or preventing a medical condition/disease which is GC-responsive mediated by the administration of GC-induced CD14 positive monocytes to a human patient.

It is also envisaged that the pharmaceutical package or kit of the present invention, further comprises means to administer GC-induced monocytes to a subject and/or buffers, vials, syringes, Teflon bags or infusion bags which are usually employed for the infusion of therapeutic agents. “Means to administer” thereby includes one or more article(s) selected from the group consisting of a syringe, a hypodermic needle, a cannula, a catheter, an infusion bag for intravenous administration, intravenous vehicles, vials, buffers, stabilizers, written instructions which aid the skilled person in the preparation of the respective doses and infusions of the invention etc.

In another embodiment, said pharmaceutical package or kit of the present invention may further comprise M-CSF.

The Mregs of the invention and/or the pharmaceutical compositions of the invention can be/are to be administered prophylactically.

Alternatively, the Mregs of the invention and/or the pharmaceutical compositions of the invention can be/are to be administered therapeutically, preferably as early as possible after the diagnosis of the respective GC-responsive disease.

The dosage regimen utilizing the Mregs/pharmaceutical composition of the present invention is selected in accordance with a variety of factors including type, species, age, weight, sex and medical condition of the patient; the severity of the condition to be treated; the route of administration etc.

In another embodiment of the present invention, the pharmaceutical compositions are used for alleviating the side-effects of a GC-therapy. Long term GC therapy is often associated with severe systemic side effects including osteoporosis with concomitant vertebral fracture; electrolyte disturbances with fluid retention; activation of Gastric ulcera; skin atrophy resulting in purpura; Striae rubrae; problems with wound healing; aseptic bone necrosis (especially Osteonecrosis of the hip); Morbus Cushing; Hypertension; Redistribution of body fat; Cataract and glaucoma; Opportunistic infections; Virilisation; Amenorrhoe; Corticosteroid Myopathy; Emotional lability (depression or hypomania); Motor restlessness; Insomnia; Iatrogenic Diabetes Mellitus; Hyperlipidemia; Flush and/or Bradycardia; to name some. For overview see: Frauman A G. An overview of the adverse reactions to adrenal corticosteroids. Adverse Drug React Toxicol Rev. 1996 Nov;15(4):203-6; or Trikudanathan S, McMahon G T. Optimum management of glucocorticoid-treated patients. Nat Clin Pract Endocrinol Metab. 2008 May;4(5):262-71. These side effects can be alleviated by way of the pharmaceutical compositions, uses and methods of the present invention. This is so, because the present invention provides for the first time a substitute GC-therapy which is characterized in that the administration of GC is replaced (either in part or in toto) with the, preferably systemic, administration of the pharmaceutical compositions/Mregs (GC-induced monocytes) of the present invention.

Thus, in a further embodiment, the present invention relates to the pharmaceutical composition or the medical use, medical methods of the present invention wherein the GC-induced monocytes substitute a GC-therapy. The present invention also relates to a GC-substitute characterized in that said substitute comprises Mregs. A “GC-substitute” or a “substitute of a GC-therapy” means a pharmaceutical composition of the present invention which is used instead of a classical GC-therapy, i.e. which replaces (in part or in toto) the systemic administration of a GC. The GC is to be administered to monocytes of a patient ex vivo, which GC-induced monocytes are than (to be) administered to that (or another) patient instead of the GC. If the GC-substitute of the present invention replaces the classical GC therapy in part, than it is envisaged that the remaining GC which is (to be) administered to the patient (in vivo) is preferably in an amount which equates with or is below an amount which does not exert side-effects in a patient. The latter term is explained herein elsewhere.

In view of the above, it becomes evident that the pharmaceutical compositions can likewise be used for the treatment of a human patient who may not be treated with GC. The term “not be treated with GC” means that said patient should not or must not receive a classical, i.e. a systemic GC-therapy (characterized by the in vivo administration of at least one GC). “Should not or must not” means that in view of the medical condition of the patient, and having regard to good clinical practice, the skilled practitioner (e.g. a physical doctor) would/should not start or would/should discontinue a classical GC-treatment, for example because the side effect of a previous GC-therapy are to severe or because other medical pre-conditions would render a GC therapy impossible or at least risky.

A “patient who may not be treated with GC” is thus a patient who suffers from severe GC-induced side effects or other medical conditions (e.g. the patient is resistant to GC-therapy—this may be the result of genetic predisposition, ongoing exposure to the cause of the inflammation (such as allergens), and pharmacokinetic disturbances (incomplete absorption or accelerated excretion or metabolism)which would prevent the skilled practitioner from the initial or further administration of a GC (preferably systemically). “GC induced side effects” are side effects which are caused by the administration (usually the systemic administration) of at least one GC. Such side effects either are or might become irreversible and include osteoporosis and concomitant vertebral fracture; Skin atrophy; Aseptic bone necrosis; and/or Cataract, to name a few. Further side effects of a GC-therapy are mentioned herein elsewhere.

The pharmaceutical composition of the present invention may thus be used for the treatment of a patient which exhibits GC-induced side effects.

In another embodiment, the present invention relates to the pharmaceutical compositions or the uses/methods of the invention for the concomitant use in the treatment of a human patient which is subject of a GC-treatment. A “human patient who is subject of a GC-treatment” is a patient which already receives or is going to receive a “classical” GC-therapy (characterized in that the GC is administered per se, preferably systemically). Such patients will significantly benefit from the concomitant administration of GC-induced monocytes because the amount of GC which is administered as such can be reduced, preferably reduced to an amount (concentration) which does not exert side-effects in a human patient.

The pharmaceutical compositions, uses or methods may also be used in the treatment of a patient that is a long-term recipient of GC-therapy and/or has developed hypersensitivity to GC treatment. Long-term treatment with GC is generally defined as a treatment of one or more months, preferably of more than two months. Long term treatment is principally needed for the clinical management of every chronic disease. Chronic diseases have been explained herein elsewhere. For an overview about long term treatment see inter alia Trikudanathan S, McMahon G T. Optimum management of glucocorticoid-treated patients. Nat Clin Pract Endocrinol Metab. 2008 May;4(5):262-71.

It is envisaged that the pharmaceutical compositions of the invention optionally comprise a GC, i.e. these pharmaceutical compositions comprise GC-induced CD14 positive monocytes and additionally at least one GC Likewise, the uses of the present invention are optionally characterized in that the GC-induced monocytes are to be administered together with at least one GC. Said GC which is (to be) administered together with the GC-induced monocytes may result (a) from the induction of the CD14 positive monocytes as such (i.e. the GC is a “left over” from the induction—the pharmaceutical compositions comprise in this embodiments residual amounts of the at least one GC—said residual amounts can be removed by washing steps if deemed expedient) and/or (b) said at least one GC is added on purpose. It is for example envisaged that the GC-induced monocytes replace a persisting GC-therapy in part (thereby lowering the amount of GC that is to be administered directly to the patient (in vivo)), i.e. at least one GC is still administered (the pharmaceutical compositions still comprise at least one GC) but that therapy is supplemented with GC-induced monocytes.

In a preferred embodiment, the amount of said at least one GC which is tolerated in a pharmaceutical composition of the invention (or in the medical uses and methods of the present invention) does not exert side-effects in a human patient. Preferably it does not exceed the threshold of 7.5 mg prednisolone equivalents/day which is known to be the threshold not to result in suppression of the hypothalamus pituitary gland axis even in long-term systemic therapy. The threshold was found empirically by clinical practice during the past decades and is generally accepted (Kaiser, H und H. K. Kley—Cortisontherapie in Klinik und Praxis—Thieme Verlag, 2002).

In another embodiment of the present invention said GC-induced monocytes and said at least one GC are to be administered simultaneously or temporary spatial.

The present invention further relates to a method of treating a GC-responsive disease in a human subject comprising the step of administering to the human subject a pharmaceutical composition as defined herein. Said human subject (patient) is explained herein elsewhere. It is envisaged that the embodiments relating to pharmaceutical compositions, the use of such compositions or the use of the Mregs of the invention for the treatment of patients apply in analogy to the methods of treatment described herein.

In another embodiment, the present invention relates to a method of treating a GC-responsive disease in a human subject comprising the step of administering ex vivo a sufficient amount of GC to human monocytes (which have been isolated from said patient) and, subsequently, administering said GC-induced monocytes to said patient or to an immunologically compatible patient (allogenic). Said GC-induced monocytes may be administered together with a GC, preferably in a concentration that does not exert side effects.

The present invention relates in a further embodiment to a teflon container comprising a glucocorticoid and optionally a colony-stimulating factor (CSF). Said teflon container is preferably designed such that it can be employed in the Miltenyi Biotech CliniMACS or MACS cell sorting system. To this end, it may comprise a connector for the attachment to the CliniMACS® Tubing Sets, preferably through a luer lock connection. Said CSF is preferably M-CSF. The amount of the at least one GC is such that it is capable of inducing CD14 positive monocytes. It is envisaged that the teflon container already comprises the GC in an amount which is sufficient to induce (GC-induce) the CD14 monocytes. Alternatively, the teflon bag already contains a first dose of GC which is such is not yet sufficient to induce the monocytes. In this embodiment, it is preferred that a second GC dose is to be administered to said teflon bag in order to end up with a total amount of GC which as such is sufficient to GC induce the CD14 positive monocytes which are to be induced/stored in that teflon bag.

Said at least one GC is preferably contained in that teflon bag in an amount which does not exert side effects in a human subject (provided that the content of said teflon bag is (to be) administered to said subject).

It is also envisaged that any the teflon bag is composed of any other suitable material which is usually employed (which is suitable) for the storage of human monocytes.

Said teflon bag may further comprise an imprint indicating that the content of said teflon bag is to be used for the GC-induction of human CD14 positive monocytes and/or comprises GC-induced human CD14 positive monocytes.

It is preferred that the teflon bag described herein is sterilized, non-pyrogenic, single-packed. It is further envisaged that it is designed such that it can comprise a fluid of volume of 100, 150, 200, 250, 300, 400, 500, 600, 700, 800, 900, 1000 or even more ml.

The present invention also relates to the teflon container as defined above further comprising CD14 positive monocytes (either GC-induced or not).

The teflon bag of the invention may be comprised by an apparatus suitable for leukapheresis.

In another embodiment, the present invention relates to a method of screening for a compound which is suitable for the treatment of a GC-responsive disease, said method comprising:
(a) contacting CD14 positive monocytes with a compound to be tested;
(b) evaluating whether the monocytes are GC-induced; and thereby
(c) identifying compounds which are suitable for the treatment of a GC-responsive disease.

The figures show:

FIG. 1: Regulation of T-cell activation by Mregs in vitro

Monocytes (1×10⁴) were co-cultured with 1×10⁵ DO11.10 OVA-TCR-tg CD4⁺ T-cells for 5 days. T-cells were stained with CFSE prior to co-culture, and on day 5 subsequently with anti-CD4 antibody. Cells were analyzed for CFSE dilution as measure of T-cell proliferation using FACS (a, b). Supernatants of co-cultures were taken on day 5 and analyzed for cytokine content using CBA technology (c-f).
a) Antigen-specific proliferation of DO11.10 CD4⁺ T-cells (1×10⁵) induced by monocytes (ratio T:Mo=10:1) for 5 days with or without 100 ng/ml OVA peptide. One representative example out of 9 is shown.
b) Percent OVA-specific proliferation of CD4⁺ T-cells is shown (mean values +/−SEM): Ctr-Mo: 31.4% +/−5.1 vs. Mreg: 17.37% +/−4.1. *p=0.0459
c) Antigen-specific IFN production of DO11.10 CD4⁺ T-cells (n=6)

Ctr-Mo: 1119 pg/ml +/−241.3

Mreg: 557 pg/ml +/−152
d) Antigen-specific IL-17 production of DO11.10 CD4⁺ T-cells (n=5)

Ctr-Mo: 24.3 pg/ml +/−5.8

Mreg: 14.2 pg/ml +/−4.4
e) Antigen-specific IL-4 production of DO11.10 CD4⁺ T-cells (n=3)

Ctr-Mo: 29.9 pg/ml +/−1.8

Mreg: 27.22 pg/ml +/−3.8
f) Antigen-specific IL-13 production of DO11.10 CD4⁺ T-cells (n=3)

Ctr-Mo: 220.9 pg/ml +/−23.5

Mreg: 199.2 pg/ml +/−14.4

FIG. 2: Mregs do not induce Treg
a) 1×10⁵ CD4⁺ T-cells from DO11.10 mice were co-cultured with either Ctr-Mo or Mregs (ratio T:Mo=10:1) for 7 days in the presence of 100 ng/ml OVA peptide, and subsequently stained for Foxp3 and CTLA-4 (intracellular) and CD4 (surface) and analyzed by FACS. Plots are representative for 5 independent experiments.
b) After depletion of CD25⁺ T-cells (using MACS technology) 1×10⁵ CFSE-labeled CD4⁺ T-cells from DO11.10 mice were co-cultured with either Ctr-Mo or Mregs (ratio T:Mo=10:1) for 7 days. Then cells were stained with CD4 antibody and proliferation of cells was analyzed by FACS. Data shown are representative of 3 independent experiments.

FIG. 3: CD80 contributes to Mreg activity

Ctr-Mo or Mregs were incubated as indicated and subsequently RNA was prepared and RT-PCR was performed using CD80-specific primer (see methods). Data represent mean and SEM of 3 independent experiments.
a) FACS analysis of surface CD80 on monocytes. Data are representative of 3 independent experiments.
b) Proliferation of CFSE-labeled CD4⁺ T-cells (1×10⁵) from DO11.10 co-cultured for 5 days with indicated monocytes (1×10⁴) and 100 ng/ml OVA peptide. Where indicated 10 μg/ml anti-CD80 antibody was used in co-culture. Cells were subsequently stained with CD4 antibody and analyzed by FACS. Dot plots shown are representative of 3 independent experiments.

FIG. 4: CD124 contributes to Mreg activity
a) Ctr-Mo or Mreg were incubated as in FIG. 3 and subsequently RNA was prepared and RT-PCR was performed using CD124-specific primer (see methods). Data represent mean and SEM of 3 independent experiments.
b) FACS analysis of surface CD124 on monocytes. Data are representative of 3 independent experiments.
c) Proliferation of CFSE-labelled CD4⁺ T-cells (1×10⁵) from DO11.10 co-cultured for 5 days with indicated monocytes (1×10⁴) and 100 ng/ml OVA peptide. Where indicated 5 μg/ml anti-CD124 antibody was used in co-culture. Cells were subsequently stained with CD4 antibody and analyzed by FACS. Dot plots shown are representative of 3 independent experiments.

FIG. 5: Mregs clinically improve colitis in a model of inflammatory bowel disease
a) Rae^{-/-} mice were injected i.v. with 1×10⁶ syngeneic CD4⁺CD25⁻ T-cells. After 20 days, when weight loss of the animals was severe, 2×10⁶ monocytes (Ctr-Mo or Mreg) were transferred i.v. and body weight of mice was measured. Body weight of animals was set 100% at the day of monocyte treatment, and weight changes were monitored over the following 9 days. Graph shows mean and SEM of 12 (Ctr-Mo) and 13 (Mregs) mice from 3 independent experiments. Student's t test: day 26 p=0.0006, day 27 p=0.005, day 28 p=0.02, and day 29 p=0.01, respectively.
b) Relative body weight of individual mice on day 26.
c) Relative body weight of individual mice on day 29.
d-g) Histology of colon (H&E staining) of mice on day 29. Images are representative for at least 5 mice of each group. d) naïve mouse, e) colitis induced, but no treatment, f) colitis induced and treatment with Mreg, g) colitis induced and treatment with Ctr-Mo. Scale on images: 200 μm.

FIG. 6: Mregs regulate CD4+ T-cell activation in vivo
a) Spleen cells of mice from colitis experiments (day 29) were CFSE-labeled and restimulated with allogeneic dendritic cells (DC) for 5 days to assess activation state of in vivo differentiated T-cells (ratio T:DC=80:1). Cells from co-culture were stained with anti-CD4 antibody and analyzed for proliferation (CFSE-dilution) using FACS. Plots show representative T cell proliferation of CD4⁺ T cells from mice of colitis experiment (see FIG. 5).
b) As in a). Summary of proliferation of CD4⁺ T-cells from 8 mice of each group from 3 independent experiments. Shown is the mean and SEM of unstimulated and restimulated (T:DC=80:1) spleen cells of colitis mice from FIG. 5. Ctr-Mo: 12.6% +/−1.7 vs. Mregs: 8.4% +/−0.9 *p=0.0449.

c-f) Supernatants of co-cultures from b) were analyzed for cytokines using CBA technology. Graphs show mean values +/−SEM (left graph) of 4-8 individual mice (right graph) from 3 independent experiments.
c) IFN: Ctr-Mo group: 976.9 pg/ml +/−77.3 vs. Mreg group: 348.5 pg/ml +/−54.4
d) IL-17: Ctr-Mo group: 105.3 pg/ml +/−28.6 vs. Mreg group: 36.6 pg/ml +/−11.0
e) IL-4: Ctr-Mo group: 145.3 pg/ml +/−52.25 vs. Mreg group: 164.5 pg/ml +/−37.7
f) IL-13: Ctr-Mo group: 275.6 pg/ml +/−54.64 vs. Mreg group: 183.8 pg/ml +/−55.03

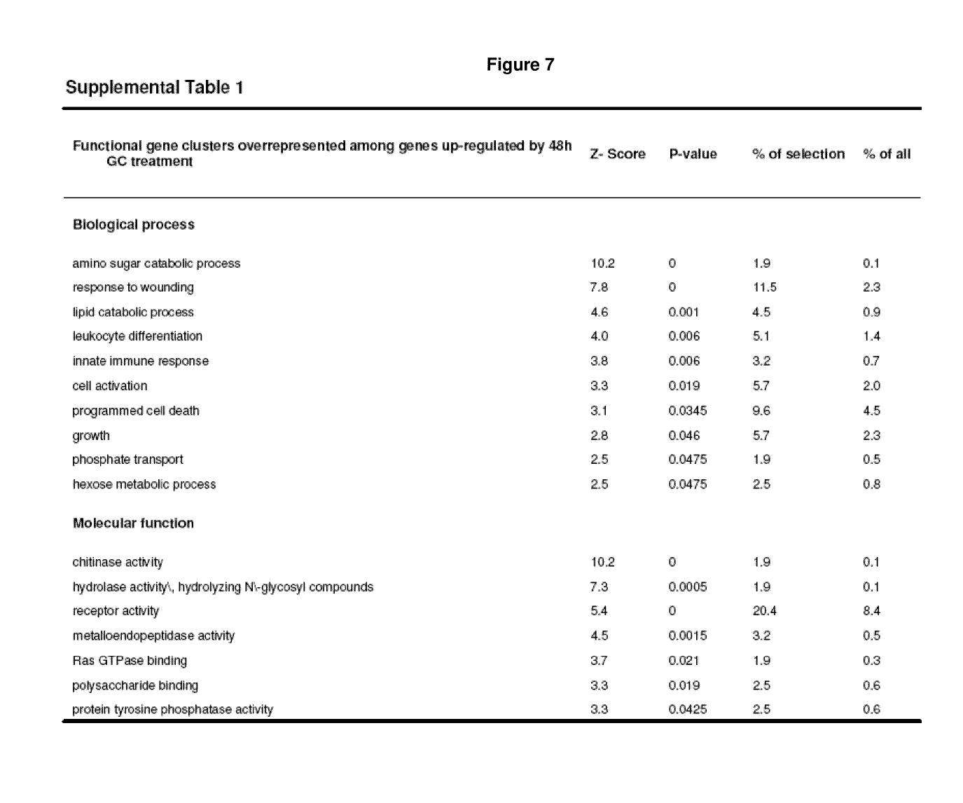

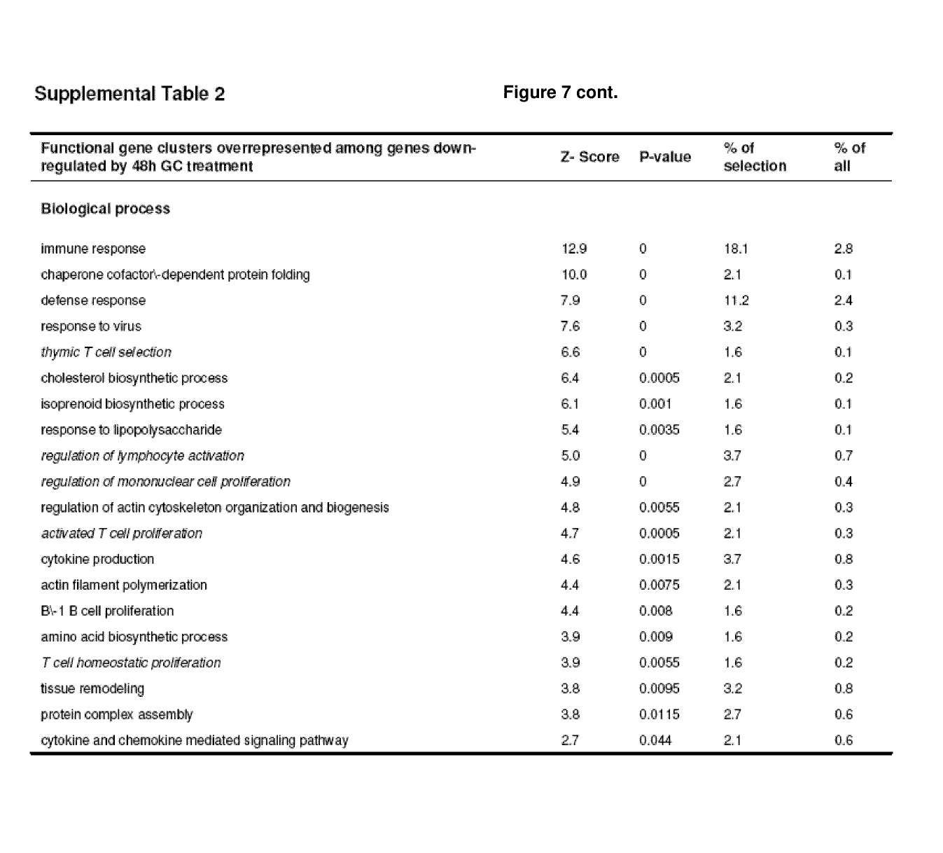

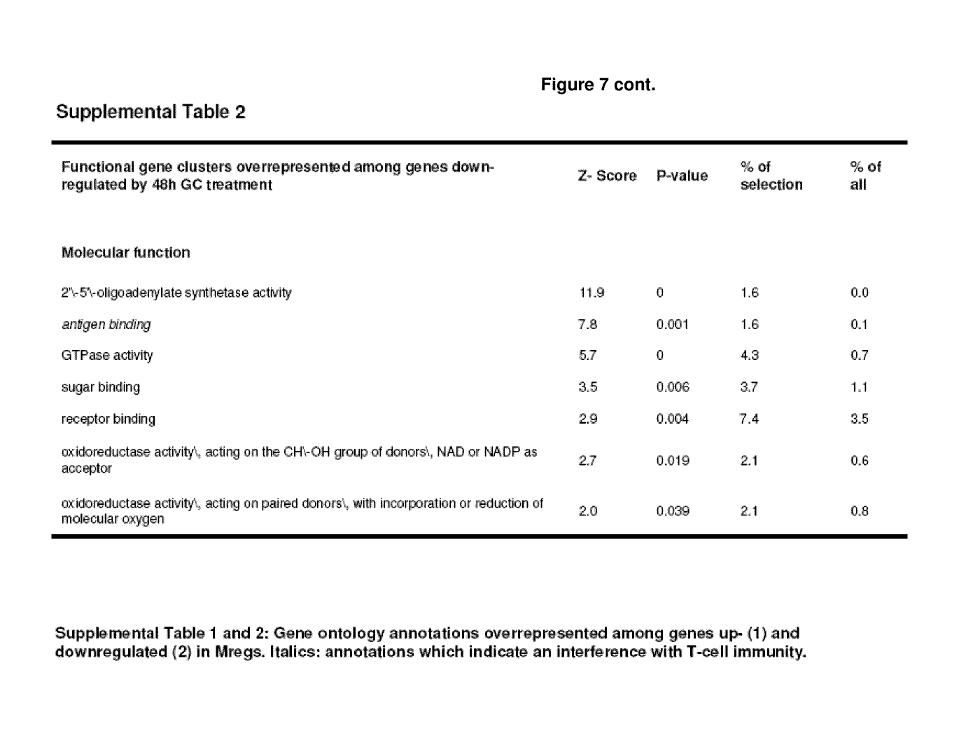

FIG. 7 (Supplemental Table 1/2): Gene ontology annotations overrepresented among genes up- (1) and downregulated (2) in Mregs. Italics: annotations which indicate an interference with T-cell immunity.

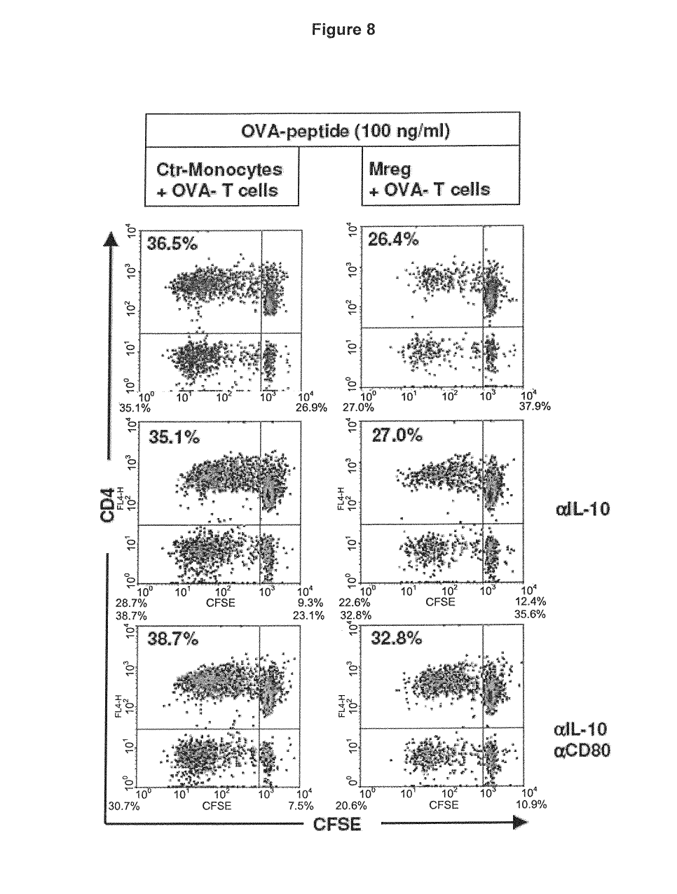

FIG. 8: IL-10 is not used by Mregs to regulate CD4+ T-cells (Suppl. FIG. 1)

Proliferation of CFSE-labeled CD4⁺ T-cells from DO11.10 co-cultured for 5 days with indicated monocytes and 100 ng/ml OVA peptide. Where indicated 10 μg/ml anti-IL-10 or anti-IL-10/anti-CD80 antibody was used in co-culture. Dot plots shown are representative of 2 independent experiments.

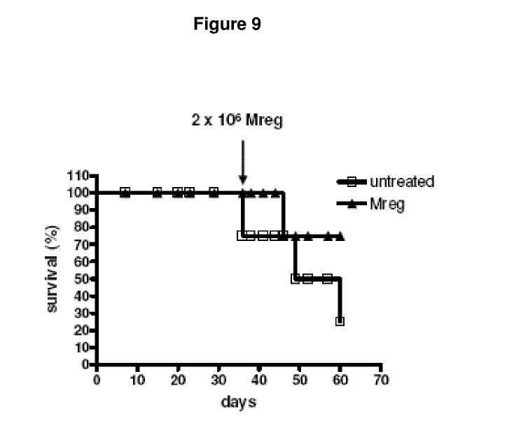

FIG. 9: Survival of severe colitis (Suppl. FIG. 2)

8 mice received 1×10⁶ CD4⁺CD25⁻ T-cells from syngeneic donors i.v. On day 39 when weight loss had significantly occurred 4 mice received 2×10⁶ syngeneic Mregs i.v. (triangle), and mice were further monitored for weight loss. 3 out of 4 mice that did not receive any treatment died by day 60 (open rectangles). Of the Mreg group 3 out of 4 mice were still alive at day 60.

This disclosure may best be understood in conjunction with the accompanying drawings, incorporated herein by references. Furthermore, a better understanding of the present invention and of its many advantages will be had from the following examples, given by way of illustration and are not intended as limiting.

### EXAMPLES

The following examples illustrate the invention. These examples should not be construed as to limit the scope of this invention. The examples are included for purposes of illustration and the present invention is limited only by the claims.

General Overview

We report here on a new and stable subtype of monocytes, induced by GC, that we named regulatory monocytes (Mreg) according to their functional property to regulate CD4⁺ T-cells. Cell surface receptors CD80 and CD124 confer their regulatory activity, but not Tregs or IL-10. Remarkably, in a mouse model of CD4⁺ T-cell-induced colitis, these monocytes are able to suppress CD4⁺ T-cell-dependent inflammation of an already established disease in vivo. Mregs could thus be target of therapeutic strategies to exploit the efficacy of GC without their adverse effects and, in this context, to support distinctly active resolution of T-cell-mediated inflammation through the innate immune system. Mregs have some similarities to so-called myeloid-derived suppressor cells (MDSC), a heterogenous group of immunosuppressive myelomonocytic cells that are strongly increased under pathological conditions such as growth of malignant tumors (17). In mice MDSC belong to the group of CD11b⁺Gr-1⁺ cells that resemble not fully differentiated monocytic/granulocytic cells (18). Mregs share with MDSC expression of CD11b, Gr-1, and CD124 (2), and both functionally suppress T-cell activation, however, in contrast to MDSC Mregs do not induce regulatory T-cells to mediate suppression (19, 23). Also the genome wide expression data (1, Suppl. Table 1 and 2, 19) clearly demonstrate that MDSCs and Mregs are distinct cell populations.

In the transgenic in vitro system, Mregs possess a residual capacity to elicit Ag-specific responses in T-cells in which they induce a qualitatively different cytokine response with markedly reduced inflammatory potential since they Ag-stimulated T-cells release IL-4 and IL-13, but very little IFN and IL-17. In contrast, Ctr-Mo elicit a T-cell population with high capacity to proliferate and to release IFN and IL-17, in addition to IL-4 and IL-13. Although we cannot detect a clear switch to e.g. Th2 differentiation, Mregs induce a specific program in T-cells, and thus are distinct regulators of CD4⁺ T-cells. It has been proposed that GCs down-regulate Th1 responses and induce a Th2 shift by a combination of direct effects on T-cells and indirect effects on APCs (24). However, this was only demonstrated for monocytes (25) and macrophages (26) activated by bacterial products; here GC inhibited IL-12 secretion which resulted in increase of Th2 responses. This is in agreement with the capacity of GCs to inhibit classical activation of macrophage by bacterial products (27, 28). In contrast to these results we now describe a direct effect of GCs on naïve monocytes which resulted in a subtype that actively modified T-cell responses.

Since inhibition of CD4⁺ T-cell proliferation was not mediated through induction of Tregs, and since presentation of antigen requires cell-cell-contacts, it is feasible to postulate that Mregs influence T-cells by a direct interaction. One mechanism that is used by the phenotypically related MDSC to inhibit e.g. IFN production and proliferation of T-cells is mediated via CD80 (29), a molecules that was significantly up-regulated also on Mregs by GC. Its neutralization abolished Mreg-induced suppression of T-cell proliferation following antigen presentation. CD80 suppresses e.g. T-cell proliferation after sufficient molecules bind to PD-L1, whose binding affinity to CD80 is much higher than to CD28, (16). As PD-L1 was equally expressed on OVA-TCR-trangenic T-cells upon co-culture with Mregs or with Ctrl-Mo, the distinct down-regulatory effects are rather linked to up-regulation and increased availability of CD80 on the surface of Mregs. In addition to CD80, the IL-4R-alpha chain (CD124) has also been described to be involved in suppressive activity of MDSC, at least towards CD8⁺ T-cells (19). Interestingly, we found that CD124 is also participating in inhibiting CD4⁺ T-cell proliferation by Mregs.

One of the anti-inflammatory effect of GCs includes their induction of apoptosis of activated T-cells in vitro and ex vivo (30, 31), but we have no evidence that this mechanism contributes to the down-regulatory effects of Mregs (data not shown).

Thus, Mregs and MDSC share two mechanisms by which they actively suppress T-cell responses, respectively.

Since DCs were discovered as professional APCs, the capacity of monocytes and macrophages to induce adaptive immune responses has received less attention.

Nonetheless, some studies have analyzed the effects of GCs on the capacity of macrophages and monocytes to induce adaptive immune responses (32-36). However, they mainly focussed on mononcytes or macrophages activated by microbial stimuli. In these cells GC inhibited up-regulation of MHC-molecules, co-stimulatory molecules and pro-inflammatory cytokines (25, 26, 32-35). Given the considerable heterogeneity of macrophages and—newly recognized—also of monocytes (3, 4, 6-12), the effects of GCs on a given cell type (e.g. classically activated macrophages) cannot be easily compared to other subtypes or stages, and especially not to naïve monocytes. Our data indicate that GC-treatment of naïve monocytes induces a subset which actively inhibits T-cell responses. We therefore termed these cells regulatory monocytes (Mreg).

The marked capability of these Mregs to suppress specific responses of CD4⁺ T cells prompted us to investigate if they are able to suppress established T-cell responses also in vivo and to induce resolution of inflammation.

Indeed, in IBD initiated by transfer of CD4⁺CD25⁻ T-cells, treatment with Mregs led to complete clinical and histological resolution of already established colitis. Remarkably, the clinical effect correlated with an inhibition of proliferation of CD4⁺ T-cells and of secretion of inflammatory cytokines IFNγ and IL-17 ex-vivo. Since Rag^{-/-} mice do not have intrinsic T-cells (37), T-cells that were regulated by Mregs were the colitogenic CD4⁺CD25⁻ T-cells, that were transferred i.v. on day 0.

Therefore Mregs have a high potential for immunotherapy of established autoimmune diseases induced or maintained by T-cells. This could make them a cornerstone of a novel therapeutic strategy for suppression of undesirable T-cell activation by actively and distinctly inducing resolution of inflammation in autoimmune diseases. Due to the similarity of murine Mregs with human GC-induced monoctyes Mregs our data suggest that these cells could be beneficial also for the treatment of human diseases. This could be of high clinical relevance since there are still many patients depending on long term, high dose GC-therapy, even despite advances in anti-inflammatory treatments. These patients often suffer from inevitable severe side effects of GC (38, 39). Since human GC-induced monocytes were shown to represent a long-living anti-inflammatory phenotype it is tempting to speculate that intermittent immunotherapy by injecting these cells or by targeting GC to monocytes could substitute, in part or even fully, for systemic GC treatment of human T cell-induced inflammation.

Mregs would then act by actively suppressing pro-inflammatory functions of effector T-cells and simultaneously by inducing resolution of inflammation (1, 40). This would present an innovative approach to dissociate the beneficial effects of GCs from their deleterious side effects.

### Example 1

### Reagents

Mice: BALB/c, DO11.10, C57BL/6, and Rag^{-/-} mice were kept under specific pathogen free (SPF) conditions, and according to federal regulations. Mice were purchased from Harlan, and used for experiments at the age of 10-12 weeks. Experiments were performed in accordance with approved protocols of the animal welfare committee of the University of Münster (Münster, Germany).

Antibodies and reagents: anti-CD4 APC (RM4-5), anti-CD80 (1G10/B7), anti-CD124 (mIL-4R-M1), and IFN-, IL-4-, IL-13-, and IL-17-FlexSets were from BD Biosciences, Heidelberg, Germany; anti-Foxp3 FITC (FJK 152) was from eBiosciences, Frankfurt, Germany, and anti-CTLA-4 PE (UC10-4B9) from Biolegend, Göttingen, Germany. CD4⁺ T cell isolation kits, and anti-CD25-, anti-CD19-, anti-CD11c-, and anti-CD90 (Thy1.2) magnetic beads were purchased from Miltenyi Biotech, Bergisch-Galdbach, Germany. CFSE was from Invitrogen, Karlsruhe, Germany. OVA peptide (323-339) was purchased from GenScript, Piscataway, N.J. USA. RPMI medium and supplemental substances were from Biochrom, Berlin, Germany. Dexamethasone was from Sigma, Taufkirchen, Germany.

### Example 2

### Generation of Mreg and Dendritic Cells (DC) From Bone Marrow

DC from bone marrow cells were isolated and generated essentially as described earlier (41, 42). For monocyte preparation, bone marrow cells were applied to a ficoll centrifugation step, and resulting interphase was deprived of CD11c⁺, CD19⁺, and CD90⁺ cells using MACS (magnetic cell sorting) technology to enrich for monocyte precursors. Monocytes were then cultured for 48 hours with 10⁻⁷M (40 ng/ml) dexamethasone in medium supplemented with M-CSF (50 ng/ml). Control monocytes (Ctr-Mo) were cultured with ethanol (1:50,000) that was the solvent for dexamethasone. After 2 days cells were washed at least 3 times and subsequently used as Mreg (dexamethasone-treated monocytes) or Ctr-Mo (ethanol-treated monocytes). In co-culture with T-cells ratio of T:Mo always was 10:1.

### Example 3

### DNA Microarray and Statistical Data Analysis

In three independent experiments, total RNA from Mregs and control monocytes was isolated and subsequently processed for microarray hybridization using Affymetrix Murine Genome 430 2.0 arrays according to the manufacturer's instructions (Affymetrix). Microarray data were analyzed by GCOS Software (Affymetrix) using data from corresponding control samples as baseline and further studied applying the Expressionist Suite software package (GeneData), which allows identification of genes that are significantly regulated in multiple independent experiments as described previously (1).

We retained only genes which were significantly regulated in every single experiment (change p-value <0.05, fold-change >2, expression over background) as well as in the complete set of experiments (fold-change of >2.0, p-value of <0.05, paired t-test).

### Example 4

### Functional Clustering

To analyze the microarray data in the context of biological functions, we used information available from the Gene Ontology (GO) consortium (http://www.geneontology.org) (1). The GO terms represent a defined vocabulary describing the biological process, cellular components, and molecular functions of genes in a hierarchical directed acyclic graph structure. Statistical analysis was performed for groups of >3 genes using GenMAPP software (13). For each of the existing GO terms, the cumulative number of genes meeting our criteria (e.g. up- or down-regulated) and of all genes represented on the microarray was calculated. The Z score is calculated for every GO term by subtracting the expected number of genes meeting the criterion from the actual number, and division of this value by the standard deviation of the actual number of genes:

$$zscore = \frac{( r - n \frac{R}{N} )}{\sqrt{n ( \frac{R}{N} ) ( 1 - \frac{R}{N} ) ( 1 - \frac{n - 1}{N - 1} )}}$$

with N as the total number of genes measured, R as the total number of genes meeting the criterion, n as the total number of genes in the specific GO term, and r as the number of genes meeting the criterion in the specific GO term. A positive Z score indicates that there are more genes meeting the criterion in the specific GO term than expected by chance. The Z-score is transferred to p-values under the assumption of a hypergeometric distribution.

### Example 5

### Isolation of T-Cells From Spleen of DO11.10 Mice, and CFSE Labelling for T-Cell Proliferation

T-cells were isolated from spleens of OVA TCR-tg DO11.10 mice as described in Ahlmann et al. (43) and purified using MACS technology with CD4⁺ isolation kit according to the manufacturer's instructions. Cells were then labelled with CFSE (0.5 μM) and employed in co-culture assays with monocytes (T:Mo=10:1) or DC (T:DC=80:1). T-cell proliferation was assessed as CFSE dilution in flow cytometry.

### Example 6

### FACS Analysis and Cytokine Measurement

FACS measurements were all performed using FACSCalibur from BD Biosciences and WinMDI 2.8 software. Antibody staining of cells was routinely done with 1 μl of the according antibody, and as described in Varga et al. (2). For intracellular staining of Foxp3 and CTLA-4, Cytoperm/Cytofix from BD Biosciences was used according to their instructions. Cytokines were determined from supernatants of co-cultures using CBA FlexSet technology from BD Biosciences, and performed according to manufacturer's instructions. Data were analyzed using FCAP Array (v1.0.1) software.

### Example 7

### Quantitative Real-Time PCR and Primers

Real-time PCR was performed as described in Ahlmann et al (43), and the following primers were used:

CD80
5′-AAA TAT GGA GAT GCT CAC GTG TCA G-3′
5′-CTG TTA TTA CTG CGC CGA ATC C-3′
CD124
5′-GCT GTC CTC CGC TCA GTT GTA G-3′
5′-CAC CCG GCA ACT GTG TTT G-3′
GAPDH:
5′-GTC CAC CAG CCT GTT GCT GTA G-3′
5′-CCC ACT CTT CCA CCT TCG ATG-3′
RPL:
5′-TGG TCC CTG CTC TCA AG-3′
5′-GGC CTT TTC CTT CCG TTT CTC-3′

### Example 8

### Transfer Colitis

Syngeneic CD4⁺ T-cells were prepared from spleen of C57BL/6 mice, and subsequently CD25⁺ cells removed using MACS technology. 1×10⁶ CD4⁺CD25⁻ T-cells were adoptively transferred into Rag^{-/-} mice (on C57BL/6 background) i.v. that do not develop functional T-cells (37). Weight of animals was monitored every two days until they lost body weight on consecutive days and colitis had established (reviewed in 22). On day 20 monocytes (2×10⁶/mouse) were transferred i.v., and weight of animals was measured for additional 9 days. On day 29 the animals were sacrificed, and colon was removed for histology. For T-cell experiments spleens were removed, and single cell suspensions prepared. Cells were CFSE-labelled and co-cultured with allogeneic DC for 5 days for restimulation. Then, T-cell proliferation and cytokine production was determined.

### Example 9

### Histology

For histological H&E stained tissue studies, colon was fixed with 4% PBS-buffered formalin using Tissue Tek Tissue Processor and Embedding Station (Miles). Sections were cut at 3- to 5-μm and mounted on superfrost slides (Fisher Scientific), deparaffinised, rehydrated, and stained with H&E (Richard Allen). Sections were mounted under Permount (Fisher Scientific) and examined by light microscopy to assess histological changes and immune cell infiltration.

### Example 10

### Statistical Analysis

Results are mean values +/−SEM. P-values are given in the figure and/or figure legends. P>0.05 were considered not to be significant. Statistical analysis was by Student's T test (two tailed and unpaired). *p<0.05, **p<0.005, ***p<0.0005

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, detailed Description, and Examples is hereby incorporated herein by reference.

### REFERENCES

Ehrchen, J. et al. Glucocorticoids induce differentiation of a specifically activated, anti-inflammatory subtype of human monocytes. Blood 109, 1265-1274 (2007).

Varga, G. et al. Glucocorticoids induce an activated, anti-inflammatory monocyte subset in mice that resembles myeloid-derived suppressor cells. J. Leukoc. Biol. 84, 644-650 (2008).

Mosser, D. M. The many faces of macrophage activation. J. Leukoc. Biol. 73, 209-212 (2003).

Gordon, S. & Taylor, P. R. Monocyte and macrophage heterogeneity. Nat. Rev. Immunol. 5, 953-964 (2005).

Wells, C. A., Ravasi, T., & Hume, D. A. Inflammation suppressor genes: please switch out all the lights. J. Leukoc. Biol. 78, 9-13 (2005).

Auffray, C., Sieweke, M. H., & Geissmann, F. Blood Monocytes: Development, Heterogeneity, and Relationship with Dendritic Cells. Annu. Rev. Immunol. (2009).

Gordon, S. Alternative activation of macrophages. Nat. Rev. Immunol. 3, 23-35 (2003).

Mosser, D. M. & Edwards, J. P. Exploring the full spectrum of macrophage activation. Nat. Rev. Immunol. 8, 958-969 (2008).

Biermann, H. et al. Murine leukocytes with ring-shaped nuclei include granulocytes, monocytes, and their precursors. J. Leukoc. Biol. 65, 217-231 (1999).

Geissmann, F., Jung, S., & Littman, D. R. Blood monocytes consist of two principal subsets with distinct migratory properties. Immunity. 19, 71-82 (2003).

Sunderkotter, C. et al. Subpopulations of mouse blood monocytes differ in maturation stage and inflammatory response. J. Immunol. 172, 4410-4417 (2004).

Strauss-Ayali, D., Conrad, S. M., & Mosser, D. M. Monocyte subpopulations and their differentiation patterns during infection. J. Leukoc. Biol. 82, 244-252 (2007).

Dahlquist, K. D., Salomonis, N., Vranizan, K., Lawlor, S. C., & Conklin, B. R. GenMAPP, a new tool for viewing and analyzing microarray data on biological pathways. Nat. Genet. 31, 19-20 (2002).

Brem-Exner, B. G. et al. Macrophages driven to a novel state of activation have anti-inflammatory properties in mice. J. Immunol. 180, 335-349 (2008).

Sakaguchi, S., Yamaguchi, T., Nomura, T., & Ono, M. Regulatory T cells and immune tolerance. Cell 133, 775-787 (2008).

Butte, M. J., Keir, M. E., Phamduy, T. B., Sharpe, A. H., & Freeman, G. J. Programmed death-1 ligand 1 interacts specifically with the B7-1 costimulatory molecule to inhibit T cell responses. Immunity. 27, 111-122 (2007).

Sica, A. & Bronte, V. Altered macrophage differentiation and immune dysfunction in tumor development. J. Clin. Invest 117, 1155-1166 (2007).

Gabrilovich, D. I. & Nagaraj, S. Myeloid-derived suppressor cells as regulators of the immune system. Nat. Rev. Immunol. 9, 162-174 (2009).

Gallina, G. et al. Tumors induce a subset of inflammatory monocytes with immunosuppressive activity on CD8+ T cells. J. Clin. Invest 116, 2777-2790 (2006).

Powrie, F., Carlino, J., Leach, M. W., Mauze, S., & Coffman, R. L. A critical role for transforming growth factor-beta but not interleukin 4 in the suppression of T helper type 1-mediated colitis by CD45RB(low) CD4+ T cells. J. Exp. Med. 183, 2669-2674 (1996).

Mudter, J., Wirtz, S., Galle, P. R., & Neurath, M. F. A new model of chronic colitis in SCID mice induced by adoptive transfer of CD62L+ CD4+ T cells: insights into the regulatory role of interleukin-6 on apoptosis. Pathobiology 70, 170-176 (2002).

Strober, W., Fuss, I. J., & Blumberg, R. S. The immunology of mucosal models of inflammation. Annu. Rev. Immunol. 20, 495-549 (2002).

Huang, B. et al. Gr-1+CD115+ immature myeloid suppressor cells mediate the development of tumor-induced T regulatory cells and T-cell anergy in tumor-bearing host. Cancer Res. 66, 1123-1131 (2006).

Elenkov, I. J. Glucocorticoids and the Th1/Th2 balance. Ann. N. Y Acad. Sci. 1024, 138-146 (2004).

Blotta, M. H., DeKruyff, R. H., & Umetsu, D. T. Corticosteroids inhibit IL-12 production in human monocytes and enhance their capacity to induce IL-4 synthesis in CD4+ lymphocytes. J. Immunol. 158, 5589-5595 (1997).

DeKruyff, R. H., Fang, Y., & Umetsu, D. T. Corticosteroids enhance the capacity of macrophages to induce Th2 cytokine synthesis in CD4+ lymphocytes by inhibiting IL-12 production. J. Immunol. 160, 2231-2237 (1998).

Ma, W. et al. Dexamethasone inhibits IL-12p40 production in lipopolysaccharide-stimulated human monocytic cells by down-regulating the activity of c-Jun N-terminal kinase, the activation protein-1, and NF-kappa B transcription factors. J. Immunol. 172, 318-330 (2004).

Ogawa, S. et al. Molecular determinants of crosstalk between nuclear receptors and toll-like receptors. Cell 122, 707-721 (2005).

Yang, R. et al. CD80 in immune suppression by mouse ovarian carcinoma-associated Gr-1+CD11b+ myeloid cells. Cancer Res. 66, 6807-6815 (2006).

Lanza, L. et al. Prednisone increases apoptosis in in vitro activated human peripheral blood T lymphocytes. Clin. Exp. Immunol. 103, 482-490 (1996)

Kirsch, A. H. et al. Apoptosis of human T-cells: induction by glucocorticoids or surface receptor ligation in vitro and ex vivo. J. Biol. Regul. Homeost. Agents 13, 80-89 (1999).

Celada, A., McKercher, S., & Maki, R. A. Repression of major histocompatibility complex IA expression by glucocorticoids: the glucocorticoid receptor inhibits the DNA binding of the X box DNA binding protein. J. Exp. Med. 177, 691-698 (1993).

Girndt, M., Sester, U., Kaul, H., Hunger, F., & Kohler, H. Glucocorticoids inhibit activation-dependent expression of costimulatory molecule B7-1 in human monocytes. Transplantation 66, 370-375 (1998).

Le, T. Y. et al. Monocyte human leukocyte antigen-DR transcriptional downregulation by cortisol during septic shock. Am. J. Respir. Crit Care Med. 169, 1144-1151 (2004).

Schwiebert, L. M., Schleimer, R. P., Radka, S. F., & Ono, S. J. Modulation of MHC class II expression in human cells by dexamethasone. Cell Immunol. 165, 12-19 (1995).

Kodelja, V. et al. Differences in angiogenic potential of classically vs alternatively activated macrophages. Immunobiology 197, 478-493 (1997).

Mombaerts, P. et al. RAG-1-deficient mice have no mature B and T lymphocytes. Cell 68, 869-877 (1992).

DeKruyff, R. H., Fang, Y., & Umetsu, D. T. Corticosteroids enhance the capacity of macrophages to induce Th2 cytokine synthesis in CD4+ lymphocytes by inhibiting IL-12 production. J. Immunol. 160, 2231-2237 (1998).

Trikudanathan, S. & McMahon, G. T. Optimum management of glucocorticoid-treated patients. Nat. Clin. Pract. Endocrinol. Metab 4, 262-271 (2008).

Demerjian, M. et al. Activators of PPARs and LXR decrease the adverse effects of exogenous glucocorticoids on the epidermis. Exp. Dermatol. (2009).

Perretti, M. & D'Acquisto, F. Annexin A1 and glucocorticoids as effectors of the resolution of inflammation. Nat. Rev. Immunol. 9, 62-70 (2009).

Krummen, M. B. et al. Effect of pimecrolimus vs. corticosteroids on murine bone marrow-derived dendritic cell differentiation, maturation and function. Exp. Dermatol. 15, 43-50 (2006).

Varga, G. et al. Active MAC-1 (CD11b/CD18) on DCs inhibits full T-cell activation. Blood 109, 661-669 (2007).

Ahlmann, M. et al. The cyclic AMP response element modulator { alpha} suppresses CD86 expression and APC function. J. Immunol. 182, 4167-4174 (2009).

1. A pharmaceutical composition adapted for use in the treatment of a disease in a patient which is a glucocorticoid (GC)-responsive disease, wherein a GC-responsive disease is a disease treatable, amelioratable and/or preventable by a GC, comprising:
GC-induced human monocytes, and optionally a pharmaceutically acceptable carrier, wherein the GC-induced monocytes are autologous or allogenic to the patient, and are characterized by:
(i) CD163 positive on mRNA and/or protein level, preferably at the protein level
(ii) CD121b positive on mRNA and/or protein level, preferably at the protein level
(iii) CD11b positive on mRNA and/or protein level, preferably at the protein level
(iv) induction of CD80 mRNA and/or increased, relative to the non-GC-induced cells, expression of CD80 protein on the cell surface by at least 5%
(v) upregulation, relative to the non-GC-induced cells, of the IL4-receptor alpha chain (CD124) on protein level by at least 5%.

2. The pharmaceutical composition according to claim 1, further comprising a GC.

3. A pharmaceutical composition adapted for use in the treatment of a disease in a patient which is a glucocorticoid (GC)-responsive disease, wherein a GC-responsive disease is a disease treatable, amelioratable and/or preventable by a GC, comprising:
human monocytes which have been induced by a GC ex vivo, and optionally a pharmaceutically acceptable carrier, wherein the GC-induced monocytes are autologous or allogenic to the patient, and are characterized by:
(i) CD163 positive on mRNA and/or protein level, preferably at the protein level
(ii) CD121b positive on mRNA and/or protein level, preferably at the protein level
(iii) CD11b positive on mRNA and/or protein level, preferably at the protein level
(iv) Induction of CD80 mRNA and/or increased, relative to the non-GC-induced cells, expression of CD80 protein on the cell surface by at least 5%
(v) Upregulation, relative to the non-GC-induced cells, of the IL4-receptor alpha chain (CD124) on protein level by at least 5%.

4. The pharmaceutical composition of claim 1, wherein at least 50% of the monocytes are GC-induced.

5. The pharmaceutical composition of claim 1, wherein the GC-induced monocytes are characterized by at least one of the following characteristics:
(i) CX3CR1^{low}—down-regulation of mRNA and/or protein—preferably at the protein level;
(ii) IL10 positive—up-regulation of mRNA;
(iii) CD38 positive—up-regulation of mRNA; and/or protein level, preferably at the protein level by at least 5%.
(iv) CCR2low—down-regulation of protein and/or mRNA, preferably at the protein level by at least 5%.

6. The pharmaceutical composition of claim 1, wherein the GC-induced monocytes are induced by a method comprising the step of contacting human monocytes ex vivo with a GC.

7. The pharmaceutical composition according to claim 2, wherein said GC is present in the pharmaceutical composition at a concentration that does not exert side-effects in a human patient

8. The pharmaceutical composition according to claim 1, wherein the GC-responsive disease is a T-cell mediated disease, an inflammatory disease and/or an autoimmune disease.

9. The pharmaceutical composition according to claim 8, wherein the GC-responsive disease is a CD4+ T-cell mediated disease.

10. The pharmaceutical composition according to claim 1, wherein the patient exhibits GC-induced side effects or is a long-term recipient of GC-therapy and/or has developed hypersensitivity to GC treatment.

11. The pharmaceutical composition of claim 8, wherein the human monocytes have been isolated from the patient and are induced ex vivo.

12. The pharmaceutical composition of claim 9, further comprising a GC.

13. A method for the preparation of a pharmaceutical composition for autologous or allogenic administration of GC-induced human monocytes to a patient adapted for the treatment of a disease in a patient which is a glucocorticoid (GC)-responsive disease, wherein a GC-responsive disease is a disease treatable, amelioratable and/or preventable by a GC, comprising:
contacting human monocytes ex vivo with a GC such that the human monocytes become/are GC-induced, wherein the GC-induced monocytes are characterized by:
(i) CD163 positive on mRNA and/or protein level, preferably at the protein level
(ii) CD121b positive on mRNA and/or protein level, preferably at the protein level
(iii) CD11b positive on mRNA and/or protein level, preferably at the protein level
(iv) Induction of CD80 mRNA and/or increased, relative to the non-GC-induced cells, expression of CD80 protein on the cell surface by at least 5%
(v) Upregulation, relative to the non-GC-induced cells, of the IL4-receptor alpha chain (CD124) on protein level by at least 5%.


SEQUENCE LISTING (8 sequences)

SEQ ID NO: 1 - DNA, 25 bp, Artificial
  Oligonucleotide derived from the CD80 gene
aaatatggagatgctcacgtgtcag

SEQ ID NO: 2 - DNA, 22 bp, Artificial
  Oligonucleotide derived from the CD80 gene
ctgttattactgcgccgaatcc

SEQ ID NO: 3 - DNA, 22 bp, Artificial
  Oligonucleotide derived from the CD124 gene
gctgtcctccgctcagttgtag

SEQ ID NO: 4 - DNA, 19 bp, Artificial
  Oligonucleotide derived from the CD124 gene
cacccggcaactgtgtttg

SEQ ID NO: 5 - DNA, 22 bp, Artificial
  Oligonucleotide derived from the GAPDH gene
gtccaccagcctgttgctgtag

SEQ ID NO: 6 - DNA, 21 bp, Artificial
  Oligonucleotide derived from the GAPDH gene
cccactcttccaccttcgatg

SEQ ID NO: 7 - DNA, 17 bp, Artificial
  Oligonucleotide derived from the RPL gene
tggtccctgctctcaag

SEQ ID NO: 8 - DNA, 21 bp, Artificial
  Oligonucleotide derived from the RPL gene
ggccttttccttccgtttctc